(19) United States
(12) Patent Application Publication
(10) Pub. No.: US 2023/0310617 A1
(43) Pub. Date: Oct. 5, 2023

(54) MICRORNA OLIGONUCLEOTIDE THERAPEUTICS FOR OVARIAN CANCER

(71) Applicant: AptamiR Therapeutics, Inc., Naples, FL (US)

(72) Inventor: Marc THIBONNIER, Naples, FL (US)

(73) Assignee: AptamiR Therapeutics, Inc., Naples, FL (US)

(21) Appl. No.: 17/656,901

(22) Filed: Mar. 29, 2022

(51) Int. Cl. A61K 47/54; A61K 47/64; A61K 47/65; C12N 15/113

(52) U.S. Cl. CPC A61K 47/545; A61K 47/64; A61K 47/542; A61K 47/65; C12N 15/113; C12N 2310/141; C12N 2310/351; C12N 2310/3513; C12N 2320/32

(57) ABSTRACT

Disclosed are novel methods and compositions to treat Ovarian Cancers and their tumor microenvironment. Compositions may include: a. One or several therapeutic agents (microRNA ONT(s)) that can modulate the growth and metastasis of Ovarian Cancer cells; b. a targeting element (e.g. folic acid, fatty acid or peptide) which binds to the Ovarian Cancer cell surface receptor FOLR1 and/or the adipocyte cell surface receptors FAT and/or FABP4; and/or c. a lipid nanoparticle carrier that enhances the intra-cellular penetration of the therapeutic agents while protecting them from degradation. The disclosure further relates to a method for targeted delivery to Ovarian Cancer cells and their tumor microenvironment of a therapeutic system to treat Ovarian Cancers in a subject in need thereof.

### SEQUENCE LISTING

The instant application contains a Sequence Listing which has been submitted in ASCII format and is hereby incorporated by reference in its entirety. Said ASCII copy, created on Mar. 24, 2022, is named APTA_P0010US Sequence Listing.txt and is 1,639 bytes in size.

### BACKGROUND

### I. Field of the Invention

The invention generally concerns compositions comprising therapeutic agents (e.g., oligonucleotide therapeutics (ONTs) such as microRNA agomirs and antagomirs) and methods for targeted delivery of such therapeutic agents to Ovarian Cancer cells and their tumor microenvironment for the treatment of human Ovarian Cancer.

### II. Description of Related Art

Ovarian Cancer is the most lethal gynecological cancer because of lack of sensitive early screening tools and frequent acquired drug resistance during treatments [1]. More than 60% of patients are diagnosed at advanced stages of the disease (Federation of Gynecology FIGO Stages III or IV) due to the ambiguous nature of the clinical signs and symptoms. In 2021, about 14,000 women died from Ovarian Cancer in the USA. Worldwide, more than 300,000 women are diagnosed with this cancer and more than 200,000 succumb to this disease every year [2, 3]. Although Ovarian Cancer is classified into more than 10 distinct histological subtypes (FIG. 1, Histologic classification of Ovarian Cancers), the most common Ovarian Cancers are Epithelial Cancers (90%), among which Serous Ovarian Cancer is the most prevalent (97%) [4-6]. High Grade Serous Ovarian Cancer (HGSOC) is the most common and deadliest type of Ovarian Cancer. Survival to 5 years is only 30% in HGSOC, 18% for patients diagnosed with stage IV tumors, with most deaths occurring within two years of diagnosis. Molecular characteristics associated with Ovarian Cancer histologic types include various mutations. HGSOC is characterized by mutations in TP53 and CCNEJ, and a high level of genetic instability, Low Grade Serous Ovarian Cancer by mutations of KRAS and BRAF, Clear Cell Carcinoma by mutations of ARID1alpha, PIK3CA, Endometroid Carcinoma by mutations of ARID1alpha, beta-catenin and PIK3CA and by PTEN loss of homozygosity [6].

Epithelial Ovarian Cancer is a multifactorial disease that cannot be easily controlled by classical therapeutic agents whose Mechanism of Action is one drug-one target or one drug-two/three targets. Due to its clinical, biological and molecular complexity, Ovarian Cancer is still considered one of the most difficult tumors to manage as it lacks a clear driver mutation [7]. Presently, debulking cytoreductive surgery represents the gold standard for the treatment of Ovarian Cancer along with platinum-based chemotherapy regimens (cisplatin or carboplatin and taxanes (paclitaxel and docetaxel)). Pharmacological treatments become ineffective over time and 80-85% of patients develop chemoresistance. For patients who become platinum resistant, few options are available and efficacy is limited for those regimens.

TABLE 1

| | | Progression Free |
|---|---|---|
| Line of Therapy | Response Rate | Survival (Months) |
| First Line | ~70% | ~19 |
| Second Line - Platinum Sensitive | ~60% | ~15 |
| Second Line - Platinum Resistant | ~30-40% | ~9 |
| Third Line - Platinum Resistant | ~15-25% | ~4-6 |

Therefore, there is an urgent need to develop novel, effective, safe, convenient and well tolerated treatment strategies for Ovarian Cancers.

Various genes have been shown to be differentially expressed in Ovarian Cancer [8, 9]. For instance, 57 Differentially Expressed Genes (DEGs) were identified between primary sites and metastases of serous Ovarian Cancer, revealing 417 up-regulated genes and 540 down-regulated genes (STRING Analysis including 514 nodes and 842 sides) [10]. NanoString data analyses of 3829 HGSOC cases from the Ovarian Tumor Tissue Analysis Consortium identified 55 genes that predicted gene-expression subtype with >95% accuracy [11].

The exchange of molecular signals leading to cell invasion and metastases is a typical feature of cancers. The shedding from the primary tumor of cancer cells and exosomes in the peritoneal cavity is a main aspect of Ovarian Cancer. Extracellular vesicles (exosomes also named oncosomes in the context of cancers) play a significant role in cell-to-cell communications and spreading of Ovarian Cancer from the primary tumor [12]. The oncosomes present in the ascites of Ovarian Cancer patients induce an invasive phenotype with immune system evasion and poor prognosis. Originating from cellular endosomes, the oncosomes contain tissue-specific signaling molecules like proteins and nucleic acids such as microRNAs which modulate the target cells phenotypes and contribute to tumor growth, angiogenesis and metastases.

MicroRNAs (miRNAs) are small non-coding RNAs that post-transcriptionally regulate genes and eventually proteins expression. miRNAs are attractive drug candidates for regulating cell fate decisions and improving complex diseases outcome because the simultaneous modulation of many target genes by a single miRNA may provide effective therapies of multifactorial diseases like Ovarian Cancer. miRNAs are differentially expressed in Ovarian Cancer and can act either as oncogenes or tumor suppressor genes [13-15]. Furthermore, miRNAs exert various effects in the Ovarian Cancer microenvironment of endothelial cells, fibroblasts, macrophages and adipocytes [16]. Therefore, miRNAs play several roles in Ovarian Cancer via the upregulation of oncogenes and/or downregulation of tumor suppressor genes, leading to:
a) the direct modulation of expression of genes involved in metabolism, proliferation, differentiation, migration, induction of angiogenesis, apoptosis and resistance to cell death of Ovarian Cancer cells,
b) the modulation of the tumor microenvironment, invasion and metastases via exosomal transfer of circulating miRNAs, and/or
c) the development of therapeutic resistance to Taxane- and Platinum-based chemotherapies [17-22].

miRNA inhibitors (“antagomirs”) are single-stranded oligonucleotides that bind to complementary miRNAs through Watson-Crick base-pairing, blocking the interaction of miRNAs with target mRNAs. miRNA mimics (“agomirs”) are chemically modified single-stranded and double-stranded oligonucleotides versions of the native miRNAs that can be loaded into the RISC complex to bind and regulate target mRNAs via their “guide” strand while the complementary “passenger” strand is degraded. The mechanisms of action of chemically modified miRNA analogs are shown in FIG. 13. To improve the structure-activity relationship of miRNA analogs, various chemical modifications can be introduced (FIG. 14).

There is a need to achieve a targeted delivery of microRNAs oligonucleotide therapeutics (miRNA ONTs) to Ovarian Cancer cells, in order to optimize their long-term efficacy and safety, improve their pharmacokinetic/pharmacodynamic profile with extended mean residence time (MRT) inside the cancer cells, reduce cost of goods, and minimize off-target effects.

### SUMMARY OF THE DISCLOSURE

As disclosed herein, cell surface receptors specifically overexpressed in tumor cells can be exploited to provide targeted delivery of miRNA ONTs to cancer cells. An example miRNA ONT structure is shown in FIG. 2.

The Folate receptor alpha (FOLR1) is a cell surface glycophosphatidylinositol (GPI)-anchored protein with a high affinity for its ligand folic acid [25]. FOLR1 is highly expressed in malignant cells, especially the Ovarian Cancer cells (FIG. 3) and has been selected as a therapeutic target and marker for the diagnosis of cancer [26, 27]. FOLR1 binds folic acid with high affinity and is involved in folate intra-cellular transport via receptor-dependent endocytosis and recycling [28]. The binding of FOLR1 ligands is followed by the invagination of the plasma membrane around the receptor-ligand conjugate, forming an endosome. Acidification of the endosome through the action of proton pumps induces the release of the ligand inside the cells. Thereafter, FOLR1 recycled to the cell surface. FOLR1 is quite relevant in gynecologic malignancy because it is dramatically overexpressed in more than 90% of Ovarian Cancers, especially in the common HGSOC type [29-32]. FOLR1 is an attractive and selective target for anticancer therapy because it is minimally expressed in normal tissues [27]. The FOLR1's selective ligand, vitamin B9 (folic acid), contains a derivatizable functional group available for conjugation to therapeutic agents. Therefore, FOLR1/folate conjugate therapy has great potential for targeted and efficient delivery of small RNAs such as miRNA ONTs to Ovarian Cancer cells.

The tumor microenvironment (TME) is the environment around a tumor, including the surrounding blood vessels, adipocytes, immune cells, fibroblasts, macrophages, signaling molecules and the extracellular matrix (ECM) [33]. The tumor and the surrounding microenvironment are closely related and interact constantly. Tumors can influence the microenvironment by releasing extracellular signals, promoting tumor angiogenesis and inducing peripheral immune tolerance, while the adipocytes and the immune cells in the microenvironment can affect the growth and evolution of cancerous cells.

Ovarian Cancers have a predilection for metastasis to the omentum, an extensive tissue layer on the surface of intra-peritoneal organs that is primarily composed of adipocytes [34, 35]. The reciprocal interplay between Ovarian Cancer cells and the adipose-rich metastatic microenvironment could be the source of new treatments for advanced Ovarian Cancers (FIG. 4). Adipocytes are an energy source for Ovarian Cancer cells and lipids and lipid transporters play a critical role in the progression of Ovarian Cancers [36-38]. The increased lipid cellular uptake by the membrane transporters Fatty Acid Translocase (FAT/CD36/SCARB3) and Fatty Acid-Binding Protein 4 (FABP4) is implicated in Ovarian Cancer metastasis [39]. FAT is expressed at a high density at the surface of human adipocytes (FIGS. 5A and 5B). FABP4 is upregulated at the adipocyte-Ovarian Cancer cells interface and is a key determinant of metastatic potential of Ovarian Cancers (FIG. 6) [40]. Therefore, lipid transporters have great potential for targeted and efficient delivery of small RNAs such as miRNA ONTs to the tumor microenvironment of Ovarian Cancers.

Described herein, in some aspects, are methods and compositions for targeted delivery of microRNA modulators (e.g., miRNA agomirs and antagomirs) to Ovarian Cancer cells and their tumor microenvironment. Such compositions and methods are useful in, for 131044428.1-7 example, optimizing long-term efficacy/safety profile, reducing cost of goods, and minimize off-target effects. In some aspects, local subcutaneous or intraperitoneal administration of formulated microRNA ONTs may be used, thus minimizing systemic exposure and “off target effects”, further improve therapeutic index, reduce cost of goods, provide patients' convenience and improved adherence and tolerance to treatment.

To achieve the goal of treating Ovarian Cancer, the present disclosure provides one or more of:
(a) Novel therapeutic agents such as miRNA ONTs (e.g., miRNA agomirs and antagomirs) capable of modulating Ovarian Cancer cell growth, proliferation and spreading;
(b) Targeting elements (e.g. folic acid, fatty acids or peptides) which bind to the Ovarian Cancer cells surface receptor FOLR1 and/or the adipocyte cells surface receptors FAT and FABP4; and
(c) Carrier or delivery nanoparticles that can deliver therapeutic agents to targeted Ovarian Cancer cells and adipocytes to enhance their intra-cellular penetration while protecting them from degradation.

Compositions that employ such therapeutic agents, targeting elements, and/or carrier or delivery nanoparticles can be used in methods employing local subcutaneous (e.g., injection, patch or microneedles) or intra-peritoneal administration of the therapeutic agents to the human Ovarian Cancer cells and their tumor microenvironment. This strategy results in minimizing systemic exposure and “off target” effects, further improving therapeutic index, reducing cost of goods, and improving patients' convenience and adherence to treatment.

Aspects of the disclosure are directed to a therapeutic agent comprising (a) a miRNA oligonucleotide therapeutic; and (b) a targeting element that binds to an ovarian cancer cell or a cell of an ovarian cancer tumor microenvironment. In some embodiments, the targeting element binds to an ovarian cancer cell (e.g., via cell surface receptor FOLR1). In some embodiments, the targeting element binds to a cell of an ovarian cancer tumor microenvironment such as an adipocyte (e.g., via cell surface receptor FAT and/or FABP4).

The therapeutic agent in the composition can be or comprise one or a combination of several miRNA agomirs and/or antagomirs targeting the sequence of one or several native miRNAs listed in Table 2. In some aspects, a composition comprises an agomir or antagomir targeting the sequence of at least, at most, or exactly 1, 2, 3, 4, 5, 6, 7, 8, 9, 10, 11, 12, 13, 14, 15, 16, 17, 18, 19, 20, 21, 22, 23, 24, or 25 of the miRNAs of Table 2, including any combination thereof. It is contemplated that any one or more of the miRNAs of Table 2 may be excluded in certain embodiments.

TABLE 2
246 miRNAs listed in alphabetic/ascending order

| | | | | |
|---|---|---|---|---|
| let-7 | miR-141 | miR-202-5p | miR-340 | miR-548ac |
| let-7a-5p | miR-142-3p | miR-203a-3p | miR-341-3p | miR-548bb-3p |
| let-7b | miR-142-5p | miR-205 | miR-342-3p | miR-548c |
| let-7d-5p | miR-143-3p | miR-206 | miR-346 | miR-548d-3p |
| let-7e | miR-144-3p | miR-208-5p | miR-3475 | miR-548h-3p |
| let-7f-3p | miR-145-5p | miR-20a-5p | miR-34a-3p | miR-548z |
| let-7g | miR-146a-5p | miR-20b-5p | miR-34a-5p | miR-551b-3p |
| let-7i-5p | miR-148a-3p | miR-21-3p | miR-363 | miR-552 |
| miR-1-3p | miR-148b-3p | miR-21-5p | miR-365 | miR-574-3p |
| miR-100 | miR-149 | miR-212 | miR-3651 | miR-574-5p |
| miR-1003 | miR-149-3p | miR-214-3p | miR-3688-5p | miR-584 |
| miR-101-3p | miR-150-5p | miR-215-5p | miR-373 | miR-590-3p |
| miR-106a | miR-151 | miR-216a | miR-375 | miR-591 |
| miR-106b | miR-152 | miR-216b | miR-376a | miR-596 |
| miR-10b | miR-153-3p | miR-217 | miR-377-3p | miR-607 |
| miR-1181 | miR-155-5p | miR-218 | miR-378 | miR-6089 |
| miR-122-5p | miR-15b-5p | miR-219-5p | miR-3784 | miR-6126 |
| miR-1228-3p | miR-16-5p | miR-22-3p | miR-381 | miR-612 |
| miR-1236 | miR-1628 | miR-221-3p | miR-382-3p | miR-613 |
| miR-124-3p | miR-17-5p | miR-222 | miR-383-5p | miR-6131 |
| miR-1246 | miR-17-92 | miR-222-5p | miR-409-3p | miR-616-3p |
| miR-1253 | miR-181-5p | miR-223 | miR-421 | miR-622 |
| miR-1254 | miR-181a | miR-224 | miR-423-3p | miR-628-5p |
| miR-125a-5p | miR-181b | miR-2353 | miR-424-5p | miR-630 |
| miR-125b-5p | miR-181d-5p | miR-23a-3p | miR-429 | miR-637 |
| miR-126-3p | miR-182-5p | miR-23b | miR-4430 | miR-654-3p |
| miR-126-5p | miR-183 | miR-24 | miR-4454 | miR-664b-5p |
| miR-1266 | miR-186-5p | miR-25 | miR-448 | miR-665 |
| miR-127-3p | miR-187 | miR-2508 | miR-450-5p | miR-7 |
| miR-1271 | miR-18b | miR-26a-5p | miR-450a | miR-708 |
| miR-1273g-3p | miR-1908 | miR-26b-5p | miR-451a | miR-718 |
| miR-128-3p | miR-191 | miR-27a-3p | miR-455 | miR-744-5p |
| miR-1287 | miR-1915 | miR-28-3p | miR-4652-3p | miR-760 |
| miR-1289 | miR-192-5p | miR-2916 | miR-484 | miR-766-3p |
| miR-129-5p | miR-1927 | miR-29a-3p | miR-489 | miR-804 |
| miR-1290 | miR-193a-3p | miR-29c-3p | miR-490-3p | miR-874-3p |
| miR-1305 | miR-193b-3p | miR-301b-3p | miR-491-5p | miR-874-5p |
| miR-1306 | miR-194 | miR-30a-5p | miR-492 | miR-891a-3p |
| miR-130a | miR-195-5p | miR-30c-3p | miR-494 | miR-9 |
| miR-130b | miR-196a-3p | miR-3135b | miR-497 | miR-92a-3p |
| miR-132 | miR-197-3p | miR-3144-3p | miR-499-3p | miR-93-5p |
| miR-133a-3p | miR-1974 | miR-3196 | miR-503-5p | miR-935 |
| miR-133b | miR-199a-5p | miR-32 | miR-504 | miR-936 |
| miR-134-3p | miR-199b-3p | miR-320d | miR-506 | miR-939 |
| miR-135a-3p | miR-19a | miR-328-3p | miR-508 | miR-96-5p |
| miR-137 | miR-19b | miR-331-3p | miR-509 | miR-99a-5p |
| miR-138-2-3p | miR-200a-3p | miR-335-5p | miR-509-3p | |
| miR-139 | miR-200b-3p | miR-338-3p | miR-520b | |
| miR-139-3p | miR-200c-3p | miR-33a-5p | miR-525-5p | |
| miR-139-5p | miR-200f | miR-33b | miR-532-5p | |

In some embodiments, the oligonucleotide therapeutic is a single-stranded oligonucleotide miRNA antagomir or agomir or a double-stranded oligonucleotide miRNA agomir. In some embodiments, the oligonucleotide therapeutic is a single-stranded oligonucleotide miRNA antagomir or agomir. In some embodiments, the single-stranded oligonucleotide therapeutic is between 7 and 23 nucleotides in length, including any range or value derivable therein.

In some embodiments, the targeting element is folic acid. In some embodiments, the folic acid is linked to the therapeutic agent.

In some embodiments, the folic acid is linked to the therapeutic agent via a spacer.

In some embodiments, the targeting element comprises a peptide having one of the following amino acid sequences:

| | | | |
|---|---|---|---|
| LSCQLYQR | CTVRTSADC | DWSSWVYRDPQT | SGVYKVAYDWQH |
| (SEQ ID | (SEQ ID | (SEQ ID NO: 3) | (SEQ ID NO: 4) |
| NO: 1) | NO: 2) | | |
| CIGNSNTLC | CTVRTSAEC | MHTAPGWGYRLS | |
| (SEQ ID | (SEQ ID | (SEQ ID NO: 7) | |
| NO: 5) | NO: 6) | | |

In some embodiments, the targeting peptide specifically binds to the Folic Acid Receptor Alpha (FOLR1).

In some embodiments, the targeting peptide is linked to the therapeutic agent.

In some embodiments, the targeting peptide is linked to the therapeutic agent via a spacer.

In some embodiments, the targeting element comprises a fatty acid having one of the following structure categorized by length:

| | |
|---|---|
| Medium Chain Fatty Acids | C10:0 Decanoic Acid |
| | C12:0 Dodecanoic Acid |
| Long Chain Fatty Acids | C16:0 Palmitic Acid |
| | C18:0 Stearic Acid |
| | C18:1 Oleic Acid |
| Very Long Chain Fatty Acids | C22:0 Docosanoic acid |
| | C32:6 Dotriacontahexaenoic Acid |
| Omega-3 Fatty Acids | C22:6 Docosahexaenoic acid |

In some embodiments, the targeting fatty acid specifically binds to the Fatty Acid Translocase (FAT/CD36/SCARB3) and/or the Fatty Acid-Binding Protein 4 (FABP4).

In some embodiments, the targeting fatty acid is linked to the therapeutic agent.

In some embodiments, the targeting fatty acid is linked to the therapeutic agent via a spacer.

In some embodiments, the therapeutic agent is linked to the targeting element by a linker selected from the group consisting of a covalent bond, a disulfide bond, a diester bond, a peptide bond, an ionic bond, and a biotin-streptavidin bond.

In some embodiments, the therapeutic agent is encapsulated within the interior of a lipid nanoparticle (LNP). In some embodiments, the therapeutic agent is associated with the surface of the LNP. In some embodiments, the therapeutic agent is associated with the exterior surface of the LNP and is excluded from the interior of the LNP. In some embodiments, one or more therapeutic agents of the disclosure are encapsulated within or associated with a LNP to enhance intra-cellular penetration of the therapeutic agent(s) while protecting them from degradation.

Also disclosed herein is a method of modulating genes expression (and consequently, in some embodiments, protein expression) in a subject comprising administering to the subject any of the compositions described above. In some embodiments, providing the composition or therapeutic agent comprises injecting the composition or therapeutic agent subcutaneously, transcutaneously, intraperitoneally or intravenously.

The method of modulating genes expression can be part of a strategy for treating a disease or condition. In some embodiments, the disease or condition is Ovarian Cancer. Accordingly, disclosed herein, in some embodiments, is a method for treating cancer such as Ovarian Cancer comprising administrating a therapeutically effective amount of a therapeutic agent of the present disclosure (e.g., one or more miRNA ONTs) to a subject in need thereof.

In some embodiments, the patient receiving the composition is or has been diagnosed with Ovarian Cancer.

### BRIEF DESCRIPTION OF THE DRAWINGS

FIG. 1 is the histologic classification of Ovarian Cancers.

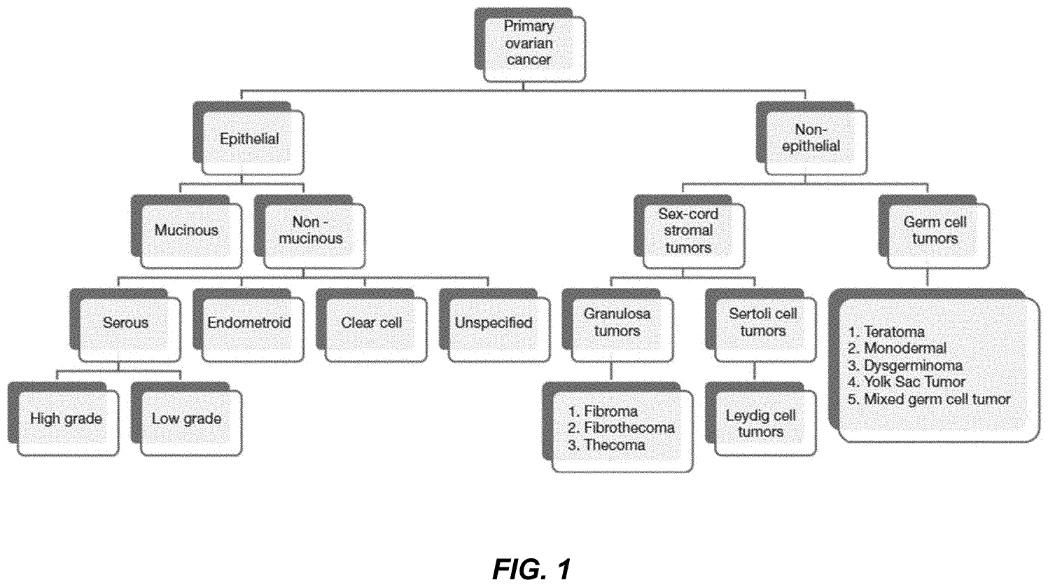

FIG. 2 shows the composition of the microRNA ONTs.

FIG. 3 illustrates the over-expression expression of the folic receptor alpha (FOLR1) in Ovarian Cancer cells.

FIG. 4 shows the interactions between Ovarian Cancer cells and adipocytes.

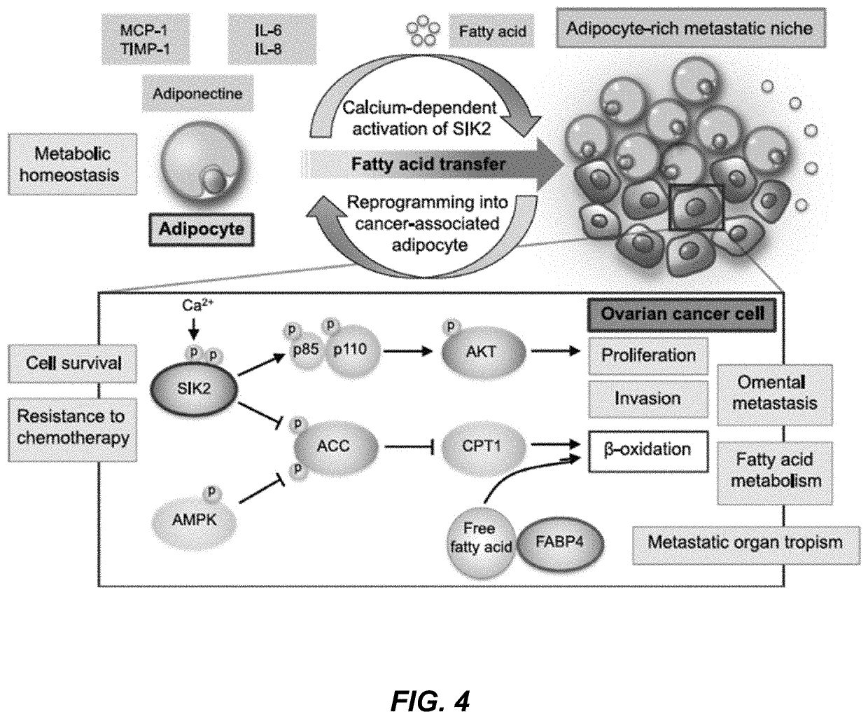

FIGS. 5A-B show the 2-D structure and the tissue expression profile of the FAT/CD36/SCARB3 membrane transporter.

FIG. 6 shows the crystal structure of the FABP4 membrane transporter.

FIG. 7 shows a Protein-Protein Interaction Network for Ovarian Cancer using the Enrichment Analysis tool STRING.

FIG. 8 shows a miRNAs-mRNAs Correlation Network in Ovarian Cancer.

FIG. 9 shows the 3-D model of a 14-mer miRNA antagomir coupled to the fatty acid docosanoic acid generated with the PyMOL program.

FIG. 10 shows 3-D models of a 12-mer (left) and a 20-mer (right) PNA antagomirs coupled to the fatty acid C16:0 palmitic acid generated by High Performance Molecular Dynamics Modeling on graphic processing units.

FIGS. 11A-C show 3-D models of an 18-mer PNA antagomir coupled to various fatty acids via a spacer containing a disulfide bond generated by High Performance Molecular Dynamics Modeling on graphic processing units.

FIG. 12 shows the 3-D model of an 18-mer PNA antagomir coupled to the peptide hexarelin via a spacer containing a disulfide bond generated by High Performance Molecular Dynamics Modeling on graphic processing units.

FIG. 13 shows a schematic of the mechanisms of action of chemically modified miRNA antagomirs and agomirs.

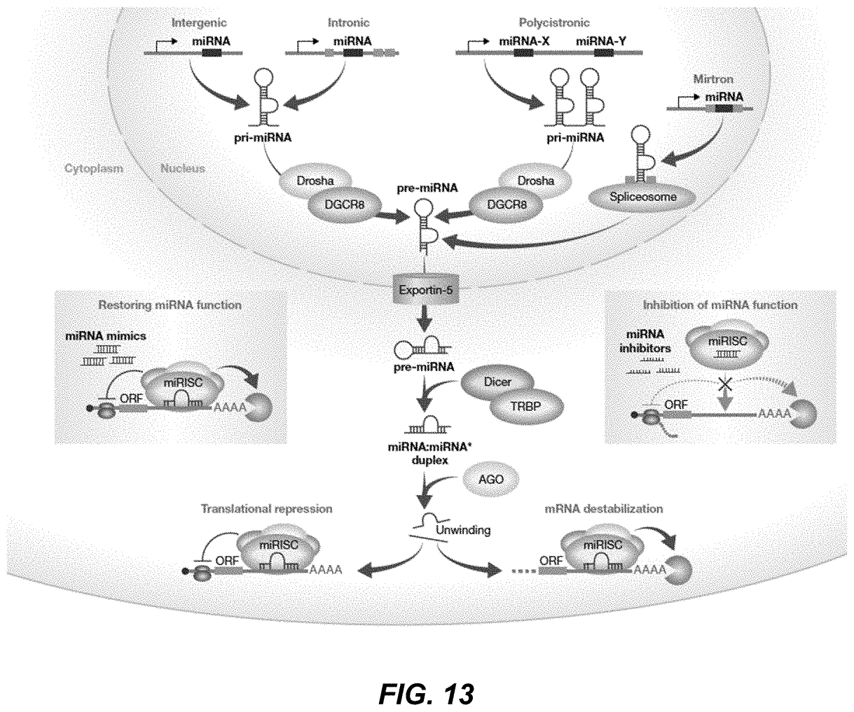

FIG. 14 shows various chemical modifications that may be introduced into an antagomir or agomir of the present disclosure.

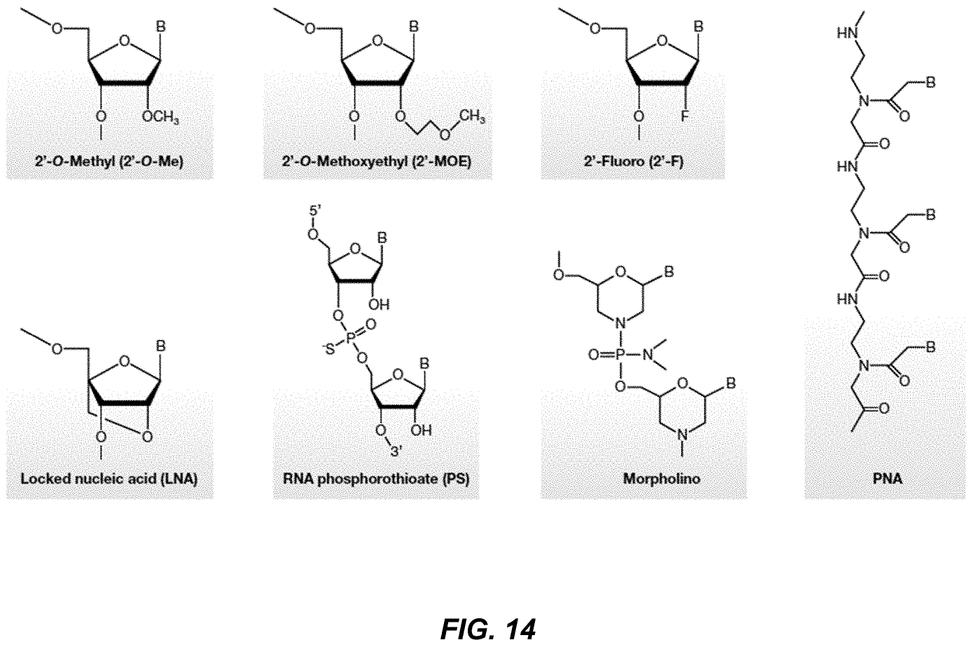

### DETAILED DESCRIPTION OF THE INVENTION

### I. Definitions

Unless otherwise defined, all technical and scientific terms used herein have the same meaning as commonly understood by one of ordinary skill in the art to which this invention belongs. In case of conflict, the present application, including definitions, will control.

As used herein, the term “miRNA analog” refers to an oligonucleotide or oligonucleotide mimetic that directly or indirectly reprograms Ovarian Cancer cells. miRNA analogs can act on a target gene or an activator or repressor of a target gene, or on a target miRNA that directly or indirectly modulates the functions of Ovarian Cancer cells.

As used herein, the term “miRNA” refers to a single-stranded oligonucleotide molecule (or a synthetic derivative thereof), which is capable of binding to a target gene (either the mRNA or the DNA) and regulating expression of that gene. In certain embodiments, the miRNA is naturally expressed in an organism.

As used herein, the term “seed sequence” refers to a 6-8 nucleotide (nt) long substring within the first 8 nt at the 5′-end of the miRNA (i.e., seed sequence) that is an important determinant of target specificity.

As used herein, the term “agomir” refers to a synthetic oligonucleotide or oligonucleotide mimetic that functionally mimics a miRNA. An agomir can be an oligonucleotide with the same or similar nucleic acid sequence to a miRNA or a portion of a miRNA. In certain embodiments, the agomir has 1, 2, 3, 4, 5, 6, 7, 8, 9 or 10 nucleotide differences from the miRNA that it mimics. Further, agomirs can have the same length, a longer length or a shorter length than the miRNA that it mimics. In certain embodiments, the agomir has the same sequence as 6-8 nucleotides at the 5′ end of the miRNA it mimics. In other embodiments, an agomir can be 7, 8, 9, 10, 11, 12, 13, 14, 15, 16, 17, 18, 19, 20, 21, 22 or 23 nucleotides in length. In certain embodiments, agomirs include any of the sequences shown in miRBase. These chemically modified synthetic RNA duplexes include a guide strand that is identical or substantially identical to the miRNA of interest to allow efficient loading into the RISC complex, whereas the passenger strand is chemically modified to prevent its loading to the Argonaute protein in the RISC complex (Thorsen S B et al., Cancer J., 18(3):275-284 (2012); Broderick J A et al., Gene Ther., 18(12):1104-1110 (2011)).

As used herein, the term “antagomir” refers to a synthetic oligonucleotide or oligonucleotide mimetic having complementarity to a specific microRNA, and which inhibits the activity of that miRNA. The term “antimir” is synonymous with the term “antagomir”. In certain embodiments, the antagomir has 1, 2, 3, 4, 5, 6, 7, 8, 9 or 10 nucleotide differences from the miRNA that it inhibits. Further, antagomirs can have the same length, a longer length or a shorter length than the miRNA that it inhibits. In certain embodiments, the antagomir hybridizes to 6-8 nucleotides at the 5′ end of the miRNA it inhibits. In other embodiments, an antagomir can be 7, 8, 9, 10, 11, 12, 13, 14, 15, 16, 17, 18, 19, 20, 21, 22 or 23 nucleotides in length. In certain embodiments, antagomirs include nucleotides that are complementary to any of the sequences shown in miRBase. Antagomirs serve as synthetic reverse complements that tightly bind to and inactivate a specific miRNA. Various chemical modifications may be used to improve nuclease resistance and binding affinity. Example modifications to increase potency include various 2′ sugar modifications, such as 2′-O-Methyl (2′-O-Me), 2′-O-methoxyethyl (2′-MOE), 2′-fluoro (2′-F) or locked nucleic acid (LNA) with a methylene bridge between the 2′ oxygen and the 4′ carbon to lock the ribose in the 3′-endo (North) conformation in the A-type conformation of nucleic acids (Lennox K A et al. Gene Ther. December 2011; 18(12):1111-1120; Bader A G et al. Gene Ther. December 2011; 18(12):1121-1126). This modification significantly increases both target specificity and hybridization properties of the molecules. The nucleic acid structure of the miRNA can also be modified by introducing Peptide Nucleic Acid (PNA) backbone modifications which make the oligonucleotide resistant to nucleases and proteases. Other modifications include 5′-(E)-Vinylphosphonate protection (5′-VP), backbone modifications (phosphorothioate (PS), Phosphorodiamidate Morpholino Oligonucleotide (PMO), Ethylene-bridged Nucleic Acid (ENA), 5-Methylcytosine modification, introduction of a “pyrimidine cassette” and/or introduction of a “DNA gap”.

As used herein, the term “interfering RNA” refers to any double stranded or single stranded RNA sequence capable of inhibiting or down regulating gene expression by mediating RNA interference. Interfering RNAs, include are not limited, to small interfering RNA (“siRNA”) and small hairpin RNA (“shRNA”). “RNA interference” refers to the selective degradation of a sequence-compatible messenger RNA transcript.

As used herein, the term “small interfering RNA” or “siRNA” refers to any small RNA molecule capable of inhibiting or down regulating gene expression by mediating RNA interference in a sequence specific manner. The small RNA can be, for example, about 16 to 21 nucleotides long.

As used herein, the term “shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA (siRNA) by a cleavage event mediated by the enzyme Dicer, which is a member of the RNase III family.

As used herein, the term “antisense oligonucleotide” refers to a synthetic oligonucleotide or oligonucleotide mimetic that is complementary to a DNA or mRNA sequence (e.g., a miRNA).

As used herein, the term “miR-mask” refers to a single stranded antisense oligonucleotide that is complementary to a miRNA binding site in a target mRNA, and that serves to inhibit the binding of miRNA to the mRNA binding site. See, e.g., Xiao, et al. “Novel approaches for gene-specific interference via manipulating actions of microRNAs: examination on the pacemaker channel genes HCN2 and HCN4,” Journal of Cellular Physiology, vol. 212, no. 2, pp. 285-292, 2007, which is incorporated herein in its entirety.

As used herein, the term “miRNA sponge” refers to a synthetic nucleic acid (e.g. a mRNA transcript) that contains multiple tandem-binding sites for a miRNA of interest, and that serves to titrate out the endogenous miRNA of interest, thus inhibiting the binding of the miRNA of interest to its endogenous targets. See, e.g., Ebert et al., “MicroRNA sponges: competitive inhibitors of small RNAs in mammalian cells,” Nature Methods, vol. 4, no. 9, pp. 721-726, 2007, which is incorporated herein in its entirety.

As used herein, the term “modulate” refers to increasing or decreasing a parameter. For example, to modulate the activity of a protein that protein's activity could be increased or decreased.

As used herein, the term “activity” refers to any measurable biological activity including, without limitation, mRNA expression or protein expression.

The “effective amount” of a composition or therapeutic agent is an amount sufficient to be effective in treating or preventing a disorder or to regulate a physiological condition in humans. In some embodiments, the disorder is cancer. In certain embodiments, the disorder is Ovarian Cancer.

A “subject” (used interchangeably herein with “patient” and “individual”) is a vertebrate, including any member of the class Mammalia, including humans, domestic and farm animals, and zoo, sports or pet animals, such as mouse, rabbit, pig, sheep, goat, cattle and higher primates.

The term “mammal” refers to any species that is a member of the class Mammalia, including rodents, primates, dogs, cats, camelids and ungulates. The term “rodent” refers to any species that is a member of the order rodentia including mice, rats, hamsters, gerbils and rabbits. The term “primate” refers to any species that is a member of the order primates, including monkeys, apes and humans. The term “camelids” refers to any species that is a member of the family camelidae including camels and llamas. The term “ungulates” refers to any species that is a member of the superorder ungulata including cattle, horses and camelids. According to some embodiments, the mammal is a human.

“Treatment”, or “treating” as used herein, is defined as the application or administration of a therapeutic agent (e.g. miRNA oligonucleotide therapeutic) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

“Pharmacogenomics”, as used herein, refers to the application of genomics technologies such as gene sequencing, statistical genetics, and gene expression analysis to drugs in clinical development and on the market More specifically, the term refers to the study of how a patient's genes determine his or her response to a drug (e.g., a patient's “drug response phenotype”, or “drug response genotype”).

### II. Regulation of Gene Expression by miRNA Agents

MicroRNAs (miRNAs) are small non-coding RNAs that bind to complementary messenger RNAs (mRNAs) and subsequently regulate genes and proteins expression [42]. Each miRNA is evolutionarily selected to modulate the expression of gene pathways. Using various open source bioinformatics software tools (e.g. TargetScan Human 8 (targetscan.org/vert 80/), metaMlR (rna.informatik.uni-freiburg.de), OncomiR (www.oncomir.org/), GeneNet package in R (strimmerlab.org/software/genets/)), 476 genes/proteins related to Ovarian Cancer were identified:

TABLE 3
476 Genes related to Ovarian Cancer listed in alphabetic order

ABCC3
ABL2
ACAP2
ACO2
ACSL4
ACTC1
ACTR1A
ACTRT3
ADAM12
ADAM17
ADAM19
ADAMDEC1
ADAMTS17
ADAMTS19
ADAMTSL1
AGO1
AKAP13
AKR1D1
AKT1
AKT2
AKT3
ALG2
ANKRD46
ANXA8L1
APAF-1
APC2
ARHGAP24
ARHGAP28
ARID1A
ARID3B
ARL5B
ASXL3
ATM
ATP5B
ATR
AURKB
AXL
B3GNT5
BAG5
BAX
BCL11B
BCL2
BCL2L1
BCR
BIRC5
BMF
BMP3
BMP4
BMP7
BNIP3
BRAF
BRCA1
BRCA2
BTLA
C10orf128
C11orf58
C1orf105
CACNA1C
CACNG8
CALR
CANX
CARD18
CASP10
CASP8
CCL5
CCN2
CCNB1
CCND1
CCND2
CCNE1
CCNG1
CCNG2
CCR2
CD1D
CD2
CD247
CD27
CD38
CD3D
CD3E
CD44
CD55
CD68
CD74
CD82
CD8A
CD97
CDC25A
CDC25B
CDH1
CDH2
CDK1
CDK12
CDK2
CDK4
CDK6
CDKN1A
CDKN2A
CEACAM1
CHEK2
CHI3L1
CHK1
CHST9
CHSY1
COBLL1
COL11A1
COL15A1
COL1A2
COL3A1
COL5A1
COL5A2
COX1
CPEB3
CPNE3
CRISPLD2
CSF1R
CSMD3
CTGF
CTNNB1
CTSK
CUL4A
CXCL1
CXCL10
CXCL11
CXCL12
CXCL9
CXCR3
CYP1B1
CYTIP
DAAM1
DCN
DCTN5
DCX
DDB2
DICER1
DKK1
DLG2
DLGAP2
DNMT1
DTD2
DVL3
E2F2
E2F3
E2F5
EBF1
EFEMP1
EGFR
EIF2C1
EIF5A2
ELAVL1
ELF5
ELN
EML1
EPB41L3
EPHA2
EPHA4
ERBB2
ERBB3
ERBB4
ESRRG
FAP
FAR1
FBN1
FBXO28
FCER1G
FCRL1
FGF1
FGF2
FHL2
FN1
FOSL2
FOXA2
FOXD4L1
FOXF2
FOXM1
FOXO3
FOXP1
FUT4
FZD2
FZD6
FZD8
GAB2
GADD45B
GALNT1
GALNT14
GALR1
GCNT1
GCNT2
GCNT4
GCOM1
GCSAM
GEMIN4
GFPT2
GM2A
GNAI3
GPR12
GPR124
GPR83
GRB7
HBEGF
HDGF
HEPHL1
HEYL
HIF1A
HIF2A
HLX
HMGA1
HMGA2
HMGB1
HNRNPC
HOXA10
HOXA13
HOXA9
HOXB2
ID1
ID4
IGF1
IGF1R
IGF2BP1
IGFBPL1
IL1
IL2
IL6R
IL8
INHBA
INSR
ITGA5
ITGB1
JAG1
JAG2
JAKMIP2
KCNA5
KDR
KEAP1
KIAA0101
KIAA0513
KLF12
KLF15
KLF4
KLF9
KLLN
KRAS
LATS2
LHX6
LIMK1
LOX
LPIN1
LRRC15
LRRK2
LSG1
LUM
LZTS1
MACC1
MAP2
MAP3K1
MAP3K7
MAPK1
MAPK14
MAPK3
MCM2
MED12L
MET
MLIP
MLLT3
MMP10
MMP16
MMP2
MMP9
MSH5
MSN
MT-ND2
MTDH
MTFR1
MTHFD1
MTSS1
MUC1
MUC16
MYC
MYCBP
MYCN
MYH9
MYO5A
NEAT1
NEFL
NEFM
NEUROD1
NEUROG1
NF1
NF2
NFIX
NFKB1
NHS
NOB1
NOTCH1
NOTCH2
NOTCH3
NREP
NRP1
NRXN3
NSD1
NUAK1
OLA1
OLFML3
OVOL1
P4HA1
PAGR1
PAK2
PAPD7
PARP1
PAX7
PCDHA10
PCDHA3
PCDHA5
PCDHGA10
PCNA
PDCD6
PDE7A
PDGFRA
PDGFRB
PDHB
PDZK1IP1
PHEX
PHLDB2
PIEZO2
PIGH
PIK3CA
PKP1
PLAG1
PLD3
PLK1
PLS3
PMAIP1
POSTN
POTED
POU3F1
PPP1R2
PRDM16
PRKAA1
PROX1
PTEN
PTGDR
PTHLH
PTPN12
PTPN4
PTPRO
PWWP2A
R3HDM4
RAB11FIP3
RAB22A
RAB30
RAB5A
RACGAP1
RAD51
RAP1B
RARRES1
RASD1
RB1
RBBP8
RHOBTB3
RHOC
RNF44
ROCK1
ROCK2
RUNX1
RUNX2
RUNX3
S1PR1
SALL2
SDC1
SEMA4D
SEMA6B
SEPTIN6
SET
SGCD
SHROOM2
SIK1
SIK2
SIRT1
SIT1
SIX2
SKAP2
SLA2
SLAMF7
SLAMF8
SLC24A4
SLC2A3
SLC31A1
SLC43A2
SLC4A4
SLC7A6
SLP1
SMAD4
SMAD7
SMTNL2
SMURF1
SMYD1
SNAI1
SNAI2
SOCS1
SOCS2
SOD2
SOS2
SOX11
SOX12
SOX4
SOX9
SPARC
SPHK1
SPSB4
SRC
SREBF1
SREBF2
SRSF1
ST7L
STAT3
STK24
STK4
STMN2
STX17
STXBP4
SYNCRIP
TAGLN
TAP1
TCF21
TCF4
TCF7L1
TEX261
TGFB1
THBS2
TIMM17A
TIMMDC1
TIMP2
TIMP3
TLN1
TLR4
TMEM239
TMEM45A
TP53
TP53I11
TRIM2
TRIM27
TRIM31
TRIM52
TSC1
TTC14
TUBB3
TWIST
TWIST1
UPA
VATIL
VCAN
VEGFA
VEGFB
VEGFC
VIM
VTN
WDR17
WNT1
WNT5A
WSCD1
XIAP
XXYLT1
YAP1
YOD1
YY1
ZEB1
ZEB2
ZNF107
ZNF138
ZNF181
ZNF346
ZNF423
ZNF485
ZNF521
ZNF697
ZNF706

An oligonucleotide therapeutic (ONT) of the present disclosure, in some embodiments, is an oligonucleotide targeting a mRNA expressed by a gene of Table 3.

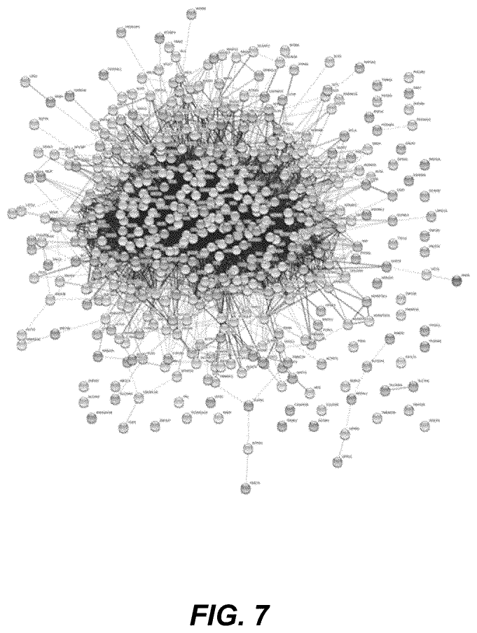

An enriched Protein-Protein Interactions network was built for Ovarian Cancer using the analysis tool String (string-db.org/) (FIG. 7).

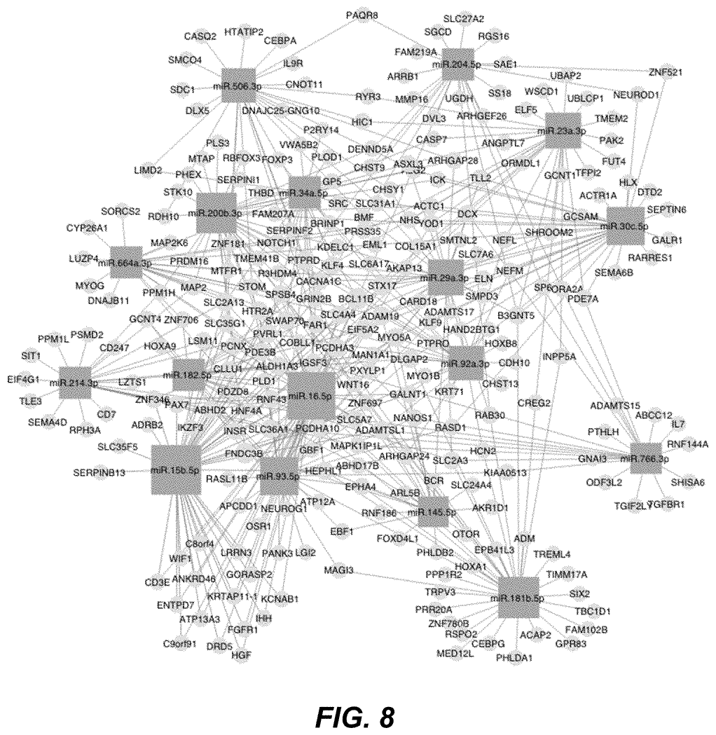

Furthermore, miRNA-miRNA and miRNA-metabolite correlation networks were built with the analysis tool Cytoscape (cytoscape.org/). For a chosen set of 17 miRNAs which are linked to Ovarian Cancer (FIG. 8), two main types of miRNAs were found in the context of Ovarian Cancer:
1. miRNAs like miR-23a-3p, miR-30c-5p, miR-145-5p, miR181b-5p, miR-214-3p, miR-506-3p, miR-664a-3p and miR-766-3p which are each connected to a specific set of targets.
2. miRNAs like miR-15a-5p, miR-16-5p, miR-29a-3p, miR-34a-5p, miR-92a-3p, miR-93-5p, miR-145-5p, miR-182-5p and miR-200b-3p which share networks of common targets.

miRNAs are synthesized as long single-stranded RNAs (pri-miRNA) that fold into hairpin loop structures (pre-miRNA). These hairpins are processed by the enzymes drosha and dicer into double-stranded mature miRNAs. The guide strand complementary to target mRNA transcripts is loaded into argonaute (AGO) proteins while the passenger strand is removed [43]. The guide strand/AGO complex then binds by sequence complementarity to targets that are typically located within 3′-untranslated regions (3′-UTR) of mRNAs.

miRNA inhibitors (antagomirs) are engineered single-stranded oligonucleotides that bind to complementary miRNAs through Watson-Crick base-pairing, blocking their interaction with target mRNAs. To improve the structure-activity relationship of miRNA inhibitors, the following chemical modifications may be implemented. The phosphates in the backbone are replaced by phosphorothioates to inhibit nuclease degradation and promote plasma protein binding, thus extending circulation time and tissue distribution. Modifications to the 2′ carbon of the sugar group (2′-Fluor, 2′-O-methyl, 2′-methoxyethyl) and Locked Nucleic Acid (LNA) conformations are also used to inhibit nuclease degradation, increase affinity to target RNAs, and blunt the immune response to foreign DNA and RNA [44].

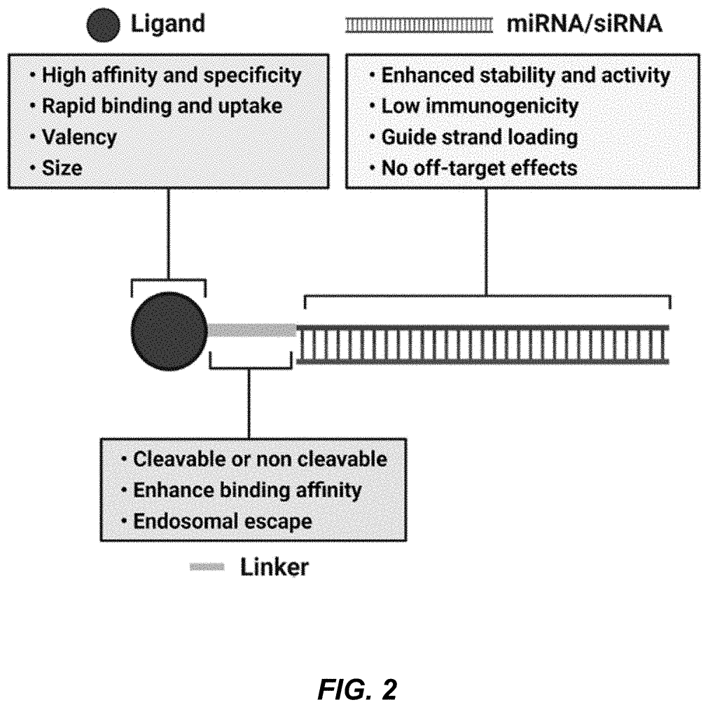

miRNA mimics (agomirs) are chemically modified versions of the native miRNAs that can be loaded into the RISC complex to bind and regulate target mRNAs via their “guide” strand while the complementary “passenger” strand is degraded. Chemical modifications are used to protect the miRNA mimic from nuclease degradation and improve potency, but the patterns of optimal chemical modification may be different from siRNA and from single-stranded miRNA inhibitors. Synthetic chemically modified single-stranded miRNAs (ss-miRNAs) can mimic the functions of double-stranded miRNAs to silence the expression of target genes [45, 46]. Such action requires the recruitment of the argonaute 2 (AGO2) protein to the target transcripts. Modified ss-miRNA mimics can combine the power of function through the RNAi pathway with the more favorable pharmacological properties of single stranded oligonucleotides. In vivo effects of ss-miRNAs in animals were achieved after systemic or local administration [45, 47, 48]. The inventors have developed targeting strategies that effectively deliver single- and double-stranded miRNAs to Ovarian Cancer cells and adipocytes (FIG. 2).

### III. Ovarian Cancer Cells Modulator Elements

In certain aspects, the compositions disclosed herein comprise therapeutic agents for modulating the fate of Ovarian Cancer cells. Exemplary Ovarian Cancer cells regulators are miRNA ONTs targeting (e.g., are an antagomir or an agomir of) one or more of miR-9, miR-15, miR-16, miR-21, miR-22, miR-23, miR-29, miR-30, miR-34, miR-92, miR-93, miR-99, miR-124, miR-125, miR-141, miR-145, miR-181, miR-182, miR-193, miR-199, miR-200, miR-205, miR-214, miR-378, miR-484, miR-506, miR-509, miR-551, miR-591, miR-664 and miR-766.

In certain embodiments, the miRNA analogs are miRNA molecules or synthetic derivatives thereof (e.g., antagomirs and agomirs). In one particular embodiment, the miRNA analog is a miRNA. miRNAs are a class of small (e.g., 18-25 nucleotides) non-coding RNAs that exist in a variety of organisms, including mammals, and are conserved in evolution. miRNAs are processed from hairpin precursors of about 70 nucleotides which are derived from primary transcripts through sequential cleavage by the RNAse III enzymes drosha and dicer. Many miRNAs can be encoded in intergenic regions, hosted within introns of pre-mRNAs or within ncRNA genes. Many miRNAs also tend to be clustered and transcribed as polycistrons and often have similar spatial temporal expression patterns. In general, miRNAs are post-transcriptional regulators that bind to complementary sequences on a target gene (mRNA or DNA), resulting in gene silencing by, e.g., translational repression or target degradation. One miRNA can target many different genes simultaneously.

Exemplary miRNA molecules targeted by the disclosed methods and compositions include without limitation those shown in Table 4 below.

TABLE 4

| | | | | |
|---|---|---|---|---|
| let-7 | miR-141 | miR-202-5p | miR-340 | miR-548ac |
| let-7a-5p | miR-142-3p | miR-203a-3p | miR-341-3p | miR-548bb-3p |
| let-7b | miR-142-5p | miR-205 | miR-342-3p | miR-548c |
| let-7d-5p | miR-143-3p | miR-206 | miR-346 | miR-548d-3p |
| let-7e | miR-144-3p | miR-208-5p | miR-3475 | miR-548h-3p |
| let-7f-3p | miR-145-5p | miR-20a-5p | miR-34a-3p | miR-548z |
| let-7g | miR-146a-5p | miR-20b-5p | miR-34a-5p | miR-551b-3p |
| let-7i-5p | miR-148a-3p | miR-21-3p | miR-363 | miR-552 |
| miR-1-3p | miR-148b-3p | miR-21-5p | miR-365 | miR-574-3p |
| miR-100 | miR-149 | miR-212 | miR-3651 | miR-574-5p |
| miR-1003 | miR-149-3p | miR-214-3p | miR-3688-5p | miR-584 |
| miR-101-3p | miR-150-5p | miR-215-5p | miR-373 | miR-590-3p |
| miR-106a | miR-151 | miR-216a | miR-375 | miR-591 |
| miR-106b | miR-152 | miR-216b | miR-376a | miR-596 |
| miR-10b | miR-153-3p | miR-217 | miR-377-3p | miR-607 |
| miR-1181 | miR-155-5p | miR-218 | miR-378 | miR-6089 |
| miR-122-5p | miR-15b-5p | miR-219-5p | miR-3784 | miR-6126 |
| miR-1228-3p | miR-16-5p | miR-22-3p | miR-381 | miR-612 |
| miR-1236 | miR-1628 | miR-221-3p | miR-382-3p | miR-613 |
| miR-124-3p | miR-17-5p | miR-222 | miR-383-5p | miR-6131 |
| miR-1246 | miR-17-92 | miR-222-5p | miR-409-3p | miR-616-3p |
| miR-1253 | miR-181-5p | miR-223 | miR-421 | miR-622 |
| miR-1254 | miR-181a | miR-224 | miR-423-3p | miR-628-5p |
| miR-125a-5p | miR-181b | miR-2353 | miR-424-5p | miR-630 |
| miR-125b-5p | miR-181d-5p | miR-23a-3p | miR-429 | miR-637 |
| miR-126-3p | miR-182-5p | miR-23b | miR-4430 | miR-654-3p |
| miR-126-5p | miR-183 | miR-24 | miR-4454 | miR-664b-5p |
| miR-1266 | miR-186-5p | miR-25 | miR-448 | miR-665 |
| miR-127-3p | miR-187 | miR-2508 | miR-450-5p | miR-7 |
| miR-1271 | miR-18b | miR-26a-5p | miR-450a | miR-708 |
| miR-1273g-3p | miR-1908 | miR-26b-5p | miR-451a | miR-718 |
| miR-128-3p | miR-191 | miR-27a-3p | miR-455 | miR-744-5p |
| miR-1287 | miR-1915 | miR-28-3p | miR-4652-3p | miR-760 |
| miR-1289 | miR-192-5p | miR-2916 | miR-484 | miR-766-3p |
| miR-129-5p | miR-1927 | miR-29a-3p | miR-489 | miR-804 |
| miR-1290 | miR-193a-3p | miR-29c-3p | miR-490-3p | miR-874-3p |
| miR-1305 | miR-193b-3p | miR-301b-3p | miR-491-5p | miR-874-5p |
| miR-1306 | miR-194 | miR-30a-5p | miR-492 | miR-891a-3p |
| miR-130a | miR-195-5p | miR-30c-3p | miR-494 | miR-9 |
| miR-130b | miR-196a-3p | miR-3135b | miR-497 | miR-92a-3p |
| miR-132 | miR-197-3p | miR-3144-3p | miR-499-3p | miR-93-5p |
| miR-133a-3p | miR-1974 | miR-3196 | miR-503-5p | miR-935 |
| miR-133b | miR-199a-5p | miR-32 | miR-504 | miR-936 |
| miR-134-3p | miR-199b-3p | miR-320d | miR-506 | miR-939 |
| miR-135a-3p | miR-19a | miR-328-3p | miR-508 | miR-96-5p |
| miR-137 | miR-19b | miR-331-3p | miR-509 | miR-99a-5p |
| miR-138-2-3p | miR-200a-3p | miR-335-5p | miR-509-3p | |
| miR-139 | miR-200b-3p | miR-338-3p | miR-520b | |
| miR-139-3p | miR-200c-3p | miR-33a-5p | miR-525-5p | |
| miR-139-5p | miR-200f | miR-33b | miR-532-5p | |

Additional miRNAs that modulate regulator molecules may be identified using publicly available Internet tools that predict miRNA targets. Modulation of a single miRNA can modulate the fate of Ovarian Cancer cells and associated adipocytes. Pathway-specific miRNAs that target multiple genes within one discrete signaling pathway are preferred, rather than universal miRNAs that are involved in many signaling pathways, functions or processes.

In a particular embodiment, the miRNA analog is an agomir. Agomirs of a particular miRNA can be identified using the screening methods disclosed herein.

In one particular embodiment, the agomir is a functional mimetic of human miR-34 which functions as a tumor suppressor by regulating the expression of several target oncogenes implicated in tumorigenesis and cancer progression [49]. miR-34a expression is decreased or lost in p53 defective cancer cells [50].

In certain embodiments, the miRNA analogs are oligonucleotide or oligonucleotide mimetics that inhibit the activity of one or more miRNAs. Examples of such molecules include, without limitation, antagomirs, interfering RNA, antisense oligonucleotides, ribozymes, miRNA sponges and miR-masks. In one particular embodiment, the miRNA analog is an antagomir. In general, antagomirs are chemically modified antisense oligonucleotides that bind to a target miRNA and inhibit miRNA function by prevent binding of the miRNA to its cognate gene target. Antagomirs can include any base modification known in the art.

In certain embodiments, the miRNA analogs are 7 to 25 nucleotides in length. One having ordinary skill in the art will appreciate that this embodies oligonucleotides having antisense portions of 7, 8, 9, 10, 11, 12, 13, 14, 15, 16, 17, 18, 19, 20, 21, 22, 23, 24 or 25 nucleotides in length, or any range there within.

In certain embodiments, the miRNA analogs are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids may be formed as composite structures of two or more oligonucleotides, modified oligonucleotides, oligonucleosides, and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

In certain embodiments, the miRNA analogs comprise at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, a basic residue or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make an oligonucleotide more resistant to nuclease digestion, thereby prolonging in vivo half-life. Specific examples of modified oligonucleotides include those comprising backbones comprising, for example, peptide nucleic acids, phosphorothioates, phosphotriesters, methyl phosphonates, short chain alkyl or cycloalkyl intersugar linkages or short chain heteroatomic or heterocyclic intersugar linkages. Particular examples are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH; amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497), each of which is herein incorporated by reference in its entirety. Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′ alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference in its entirety. Morpholino-based oligomeric compounds are known in the art described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991, each of which is herein incorporated by reference in its entirety. Cyclohexenyl nucleic acid oligonucleotide mimetics are described in Wang et al., J. Am. Chem. Soc., 2000, 122, 8595-8602, the contents of which is incorporated herein in its entirety.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having peptide nucleic acid backbone, morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety.

In certain embodiments, miRNA analogs comprise one or more substituted sugar moieties, e.g., one of the following at the 2′ position: OH, SH, SCH₃, F, OCN, OCH₃, OCH₃ O(CH₂)ₙ CH₃, O(CH₂)ₙ NH₂ or O(CH₂)ₙ CH₃ where n is from 1 to about 10; Ci to CIO lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacokinetic/pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)]. Other preferred modifications include 2′-methoxy (2′-O—CH₃), 2′-propoxy (2′—OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

In certain embodiments, miRNA analogs comprise one or more base modifications and/or substitutions. As used herein, “unmodified” or “natural” bases include adenine (A), guanine (G), thymine (T), cytosine (C) and uracil (U). Modified bases include, without limitation, bases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic bases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine (Kornberg, A., DNA Replication, W. H. Freeman & Co., San Francisco, 1980, pp 75-′7′7; Gebeyehu, G., et al. Nucl. Acids Res. 1987, 15:4513). A “universal” base known in the art, e.g., inosine, can also be included. 5-Me-C substitutions can also be included. These have been shown to increase nucleic acid duplex stability by 0.6-1.20C (Sanghvi, Y. S., in Crooke, S. T. and Lebleu, B., eds., Antisense Research and Applications, CRC Press, Boca Raton, 1993, pp. 276-278). Further suitable modified bases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

It is not necessary for all positions in a given oligonucleotide to be uniformly modified, and in fact more than one of the aforementioned modifications may be incorporated in a single oligonucleotide or even at within a single nucleoside within an oligonucleotide.

In certain embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In certain embodiments, the miRNA agent or other therapeutic agent is linked (covalently or non-covalently) to one or more moieties or conjugates that enhance the activity, cellular distribution, or cellular uptake of the oligonucleotide. Such moieties include, without limitation, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937), each of which is herein incorporated by reference in its entirety. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety.

The miRNA analogs must be sufficiently complementary to the target mRNA, i.e., hybridize sufficiently well and with sufficient specificity, to give the desired effect “Complementary” refers to the capacity for pairing, through hydrogen bonding, between two sequences comprising naturally or non-naturally occurring bases or analogs thereof. For example, if a base at one position of a miRNA analog is capable of hydrogen bonding with a base at the corresponding position of a target nucleic acid sequence, then the bases are considered to be complementary to each other at that position. In certain embodiments, 100% complementarity is not required. In other embodiments, 100% complementarity is required.

miRNA analogs for use in the methods disclosed herein can be designed using routine methods. Additional target segments are readily identifiable by one having ordinary skill in the art in view of this disclosure. Target segments of 5, 6, 7, 8, 9, 10 or more nucleotides in length comprising a stretch of at least five (5) consecutive nucleotides within the seed sequence, or immediately adjacent thereto, are considered to be suitable for targeting a gene. In some embodiments, target segments can include sequences that comprise at least the 5 consecutive nucleotides from the 5′-terminus of one of the seed sequence (the remaining nucleotides being a consecutive stretch of the same RNA beginning immediately upstream of the 5′-terminus of the seed sequence and continuing until the miRNA agent contains about 5 to about 30 nucleotides). In some embodiments, target segments are represented by RNA sequences that comprise at least the 5 consecutive nucleotides from the 3′-terminus of one of the seed sequence (the remaining nucleotides being a consecutive stretch of the same miRNA beginning immediately downstream of the 3′-terminus of the target segment and continuing until the miRNA agent contains about 5 to about 30 nucleotides). One having skill in the art armed with the sequences provided in U.S. Pat. No. 9,034,839 will be able, without undue experimentation, to identify further preferred regions to target using miRNA analogs. Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target nucleic acid sequences), to give the desired effect.

In certain embodiments, miRNA agents used in the compositions and methods disclosed herein are expressed from a recombinant vector. Suitable recombinant vectors include, without limitation, DNA plasmids, viral vectors or DNA minicircles. Generation of the vector construct can be accomplished using any suitable genetic engineering techniques well known in the art. In certain embodiments, miRNA agents are synthesized in vitro using chemical synthesis techniques.

### IV. Specific Targeting of Ovarian Cancer Cells and Associated Adipocytes

The present disclosure provides compositions and methods for targeted delivery of miRNA ONTs (e.g., miRNA antagomirs or agomirs) to Ovarian Cancer cells and/or their tumor microenvironment. Specifically, compositions and agents disclosed herein selectively deliver miRNA ONTs to Ovarian Cancer cells or their tumor microenvironment. The composition of example miRNA ONTs is shown in FIG. 2.

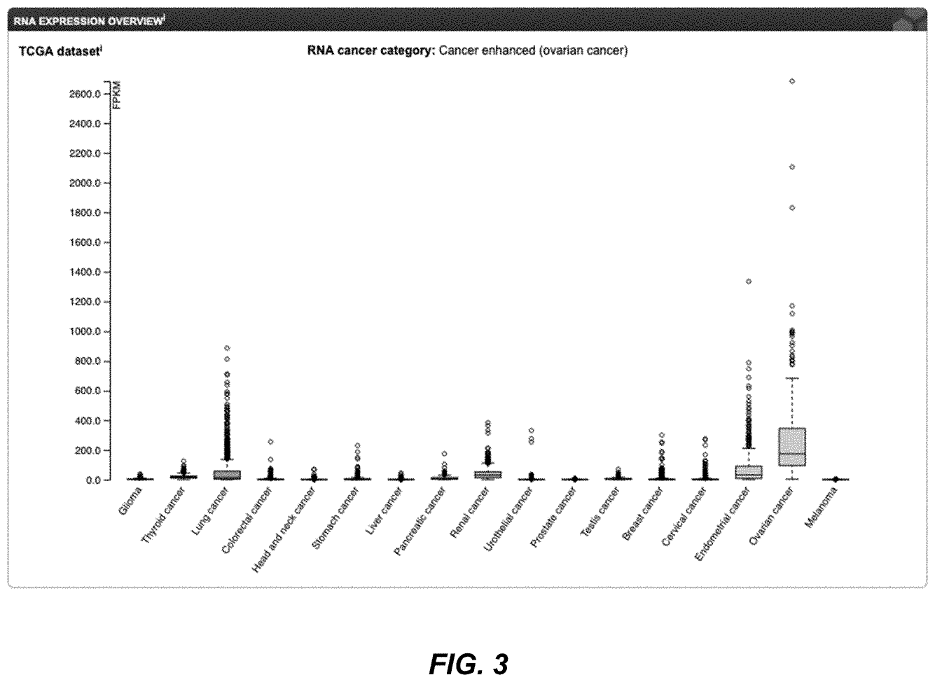

In some embodiments, the disclosed compositions bind to Ovarian Cancer target cell surface markers. An exemplary Ovarian Cancer surface marker is the Folic Acid Receptor alpha (FOLR1) which is a 37-42 kDa protein that mediates the cellular uptake of folic acid and reduced folates. FOLR1 is overexpressed at the surface of Ovarian Cancer cells (FIG. 3) The mature FOLR1 is an N-glycosylated protein that is anchored to the cell surface by a GPI linkage. FOLR1 is internalized to the endosomal system where it dissociates from its ligand before recycling to the cell surface.

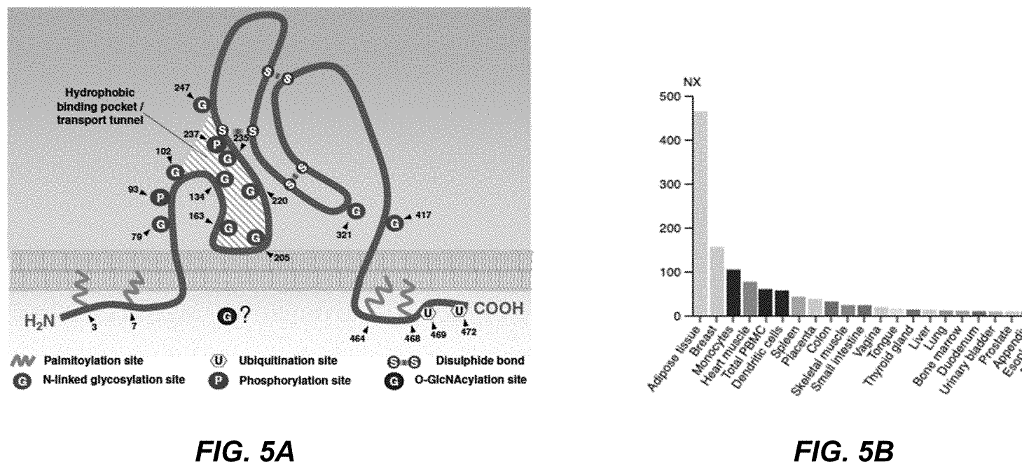

In some embodiments, compositions bind to surface receptors of Ovarian Cancer microenvironment cellular components. For example, the Fatty Acid Transporter (FAT, a.k.a CD36 or SCARB3) is an integral membrane glycoprotein made of a single chain of 472 amino acids (53 kDa) that has a hairpin membrane topology with two transmembrane spanning regions, with both the NH₂ and COOH termini as short segments in the cellular cytoplasm (FIG. 5) [51-53]. FAT is present at a very high density at the surface of human adipocytes. FAT cycles between the adipocyte membrane and intra-cellular compartments (endosomes). Accordingly, aspects of the disclosure include miRNA antagomirs or agomirs targeted to Ovarian Cancer microenvironment using an FAT-targeting agent.

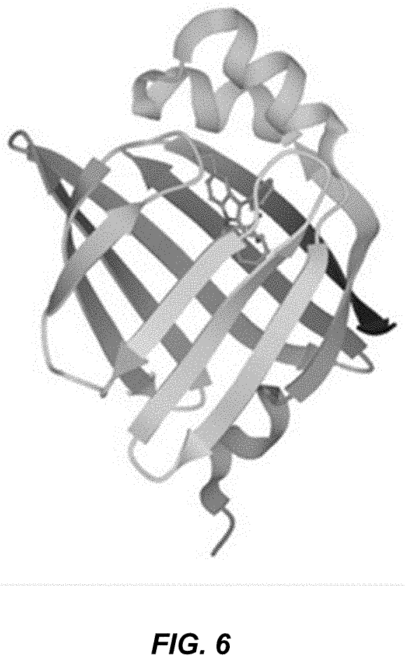

The Fatty Acid Binding Protein 4 (FABP4) is another transmembrane transporter highly expressed at the surface of human adipocytes (FIG. 6). Accordingly, aspects of the disclosure include miRNA antagomirs or agomirs targeted to Ovarian Cancer microenvironment using an FAB4-targeting agent.

Molecules that bind to adipocyte cell surface receptors/transporters may be exploited for the delivery of a variety of compositions into cells.

In some embodiments, compositions may comprise targeting elements which selectively bind one or more the above-identified markers, thus enhancing the selective delivery of miRNA ONTs to adipocytes in order to reduce or block the proliferation and metastasis of Ovarian Cancer cells. Knowledge of the cell surface markers allows for their isolation by Flow Cytometry Cell Sorting (FACS) for subsequent screening and selection of targeting agents.

miRNA ONTs may also be delivered in lipid nanoparticle (LNP) formulations. In some embodiments, LNP delivery of oligonucleotides involves encapsulation of the oligonucleotides inside a nanoparticle made of three components: structural lipids that form the lipid bilayer and maintain its rigidity; a cationic lipid to promote the incorporation of the negatively charged oligonucleotides into the particle and to facilitate escape from the endosomal pathway after cell internalization; and a “shield”, often polyethylene glycol, to increase circulation time and minimize plasma protein binding [54]. An LNP-formulated oligonucleotide can be administered subcutaneously or intra peritoneally.

### V. Examples

### Example 1: Mirna Onts with Modified Structure and Length

The disclosed miRNA ONTs are designed according to several criteria:
a. Elimination of potential toxicity by replacing PS backbone and LNA sugar chemical modifications by a PNA backbone.
b. Preservation of resistance to nucleases and proteases/peptidases degradation.
c. Avoidance of chirality.
d. Minimization of binding to circulation proteins (e.g. albumin).
e. Conjugation to a targeting agent to optimize delivery to Ovarian Cancer cells and their tumor microenvironment.
f. Optimization the Pharmacokinetic/Pharmacodynamic profile aiming at the extended Mean Residence Time of a much reduced effective dose.

These molecules, either alone (“naked”) or combined to folic acid or a short peptide or a fatty acid, are tested in models of established Epithelial Ovarian Cancer cell lines (e.g. SKOV3, SKOV3/CDDP, PA1, CAOV3, SW626, ES-2, HO-8910) as well as primary cultures of human adipocytes. Negative Control cell lines such HepG2 (liver) and A-549 VIM RFP (lung cancer) are tested too. Cellular High Content Imaging and Nanostring Gene Expression Profiling is used to assess the pharmacodynamic properties of the miRNA ONTs.

### Example 2: Folic Acid/miRNA ONTs Conjugates (“FolamiRs”)

The FOLR1/folate conjugate therapy has great potential for targeted and efficient delivery of small RNAs such as miRNA ONTs to Ovarian Cancer cells. Conjugates made of single or double stranded miRNA analogs linked to folic acid (“FolamiRs”) are synthesized. Folic acid is attached at the 3′ end or the 5′ end of miRNA analogs. Fluorescently labeled and scrambled miRNA AdipomiRs are also synthesized.

### Example 3: Fatty Acid/miRNA ONTs Conjugates (“AdipomiRs”)

Fatty acids have been used as chemical permeation enhancers (CPE) for various drugs, including oligonucleotides [55] [56]. Conjugates made of single or double stranded miRNA analogs linked to fatty acids (“AdipomiRs”) are synthesized. Fatty acids of varying lengths are attached at the 3′ end or the 5′ end of miRNA analogs. Fluorescently labeled and scrambled miRNA AdipomiRs are also synthesized. Table 5 below categorizes the fatty acids tested by length:

TABLE 5
Categorization of Fatty Acids by Length

| | |
|---|---|
| Medium Chain Fatty Acids | C10:0 Decanoic Acid |
| | C12:0 Dodecanoic Acid |
| Long Chain Fatty Acids | C16:0 Palmitic Acid |
| | (Hexadecanoic Acid) |
| | C18:0 Stearic Acid |
| | C18:1 Oleic Acid |
| Very Long Chain Fatty Acids | C22:0 Docosanoic acid |
| | C32:6 Dotriacontahexaenoic Acid |
| Omega-3 Fatty Acids | C22:6 Docosahexaenoic acid |

### Example 4: In Silico Modeling of AdipomiRs

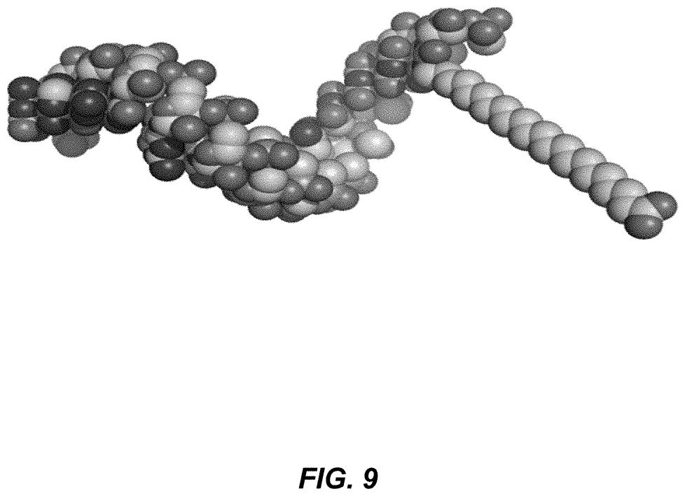

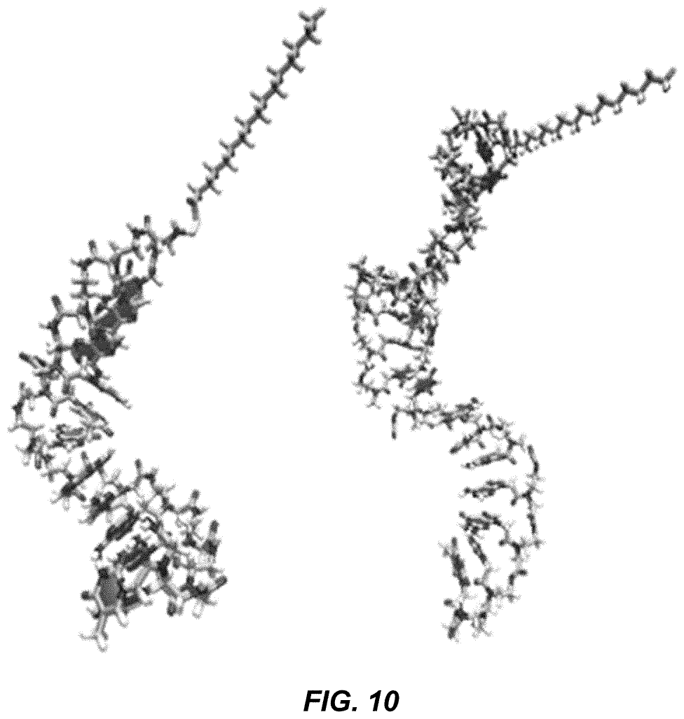

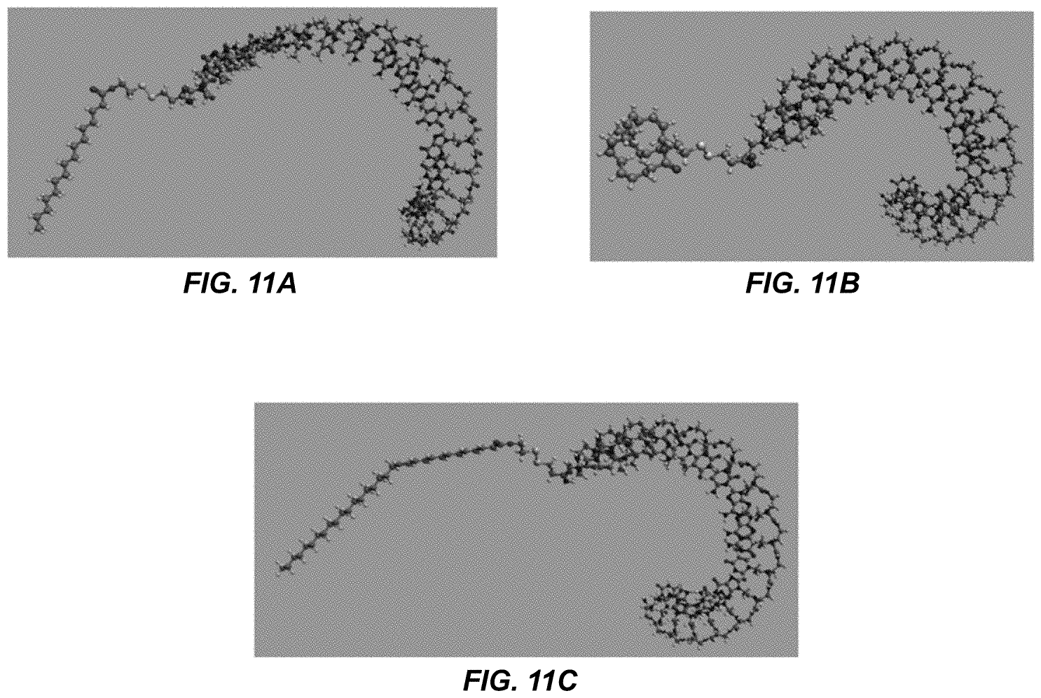

The open-source model visualization PyMOL program was used to produce 3D images of single stranded miRNA analogs conjugated to fatty acids. FIG. 9 shows the 3D model of a 14-mer miRNA antagomir coupled to the fatty acid docosanoic acid. FIG. 10 shows 3-D models of a 12-mer (left) and a 20-mer (right) PNA antagomirs coupled to the fatty acid C16:0 palmitic acid generated by High Performance Molecular Dynamics Modeling on graphic processing units. FIG. 11 shows 3-D models of an 18-mer PNA antagomir coupled to various fatty acids via a spacer containing a disulfide bond generated by High Performance Molecular Dynamics Modeling on graphic processing units.

### Example 5: Peptide/miRNA ONTs Conjugates (“PeptidomiRs”)

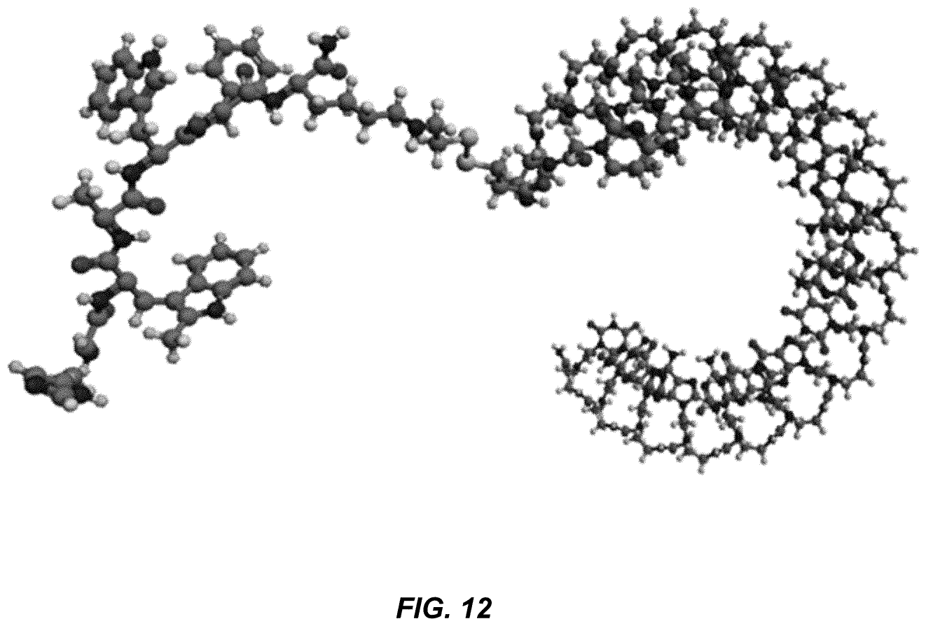

Short peptides can also be transported by FAT. Hexarelin, a chemically stable and potent Growth Hormone secretagogue (His-D-2-Me-Trp-Ala-Trp-D-Phe-Lys-NH2, Molecular Formula: C₄₇H₅₈N₁₂O₆, Molecular Weight: 887), has recently been shown to have beneficial effects on fat metabolism via the FAT/CD36 transporter [57, 58]. Conjugates made of single or double stranded miRNA analogs conjugated to a peptide (“PeptidomiRs”) are synthesized. Short peptides are attached at the 3′ end or the 5′ end of miRNA analogs. Fluorescently labeled and scrambled miRNA PeptidomiRs are also synthesized. FIG. 12 shows the 3-D model of an 18-mer PNA antagomir coupled to the peptide hexarelin via a spacer containing a disulfide bond generated by High Performance Molecular Dynamics Modeling on graphic processing units.

### Example 6: Lipid Nanoparticles/miRNA Conjugates (“LipomiRs”)

Lipid nanoparticles (LNPs) have been optimized for cellular uptake and efficient endosomal escape of siRNAs after systemic administration [59-61], but have not been extensively evaluated after local delivery to Ovarian Cancer cells and adipose tissue.

In vitro LNP delivery of a miRNA to human adipocytes: An experiment was performed with LNPs made of structural lipids, a cationic lipid, and PEG. Four different LNP formulations were used: LNP1, LNP2, LNP3, and LNP4. Mature human adipocytes in primary culture were transfected with a negative control (empty LNPs) or LNPs loaded with varying amounts (5 to 250 nM) of a double stranded miR-124 (a miRNA that is not expressed in adipocytes). Two days later, the amount of miR-124 introduced into the adipocytes and the down-regulation of target mRNAs were measured by qRT-PCR miR-124 was detected in the adipocytes in a dose-dependent fashion (RQ up to 121-fold) whereas the expression of 2 control miRNAs (let-7 and miR-143) was not modified. LNP1 and LNP2 provided the most efficient delivery of miRNA, LNP3 provided an intermediate level of efficiency, and LNP4 was relatively inefficient. The expression of 3 target genes of miR-124 (CD164, IQGAP1 and VAMP3) was knocked down in a dose-dependent fashion whereas the expression of 2 control genes (FABP4 and leptin) was not modified.

SDC Liposome formulations: Sphingomyelin is the most abundant phospholipid (40%) of the human adipocyte membrane. Sphingomyelin combines with cholesterol to form lipid rafts that are involved in many cell processes, such as membrane sorting and trafficking, signal transduction, and cell polarization [62, 63]. Sphingomyelin/cholesterol liposomes have greater stability than DSPC/cholesterol liposomes and can deliver more efficiently entrapped drugs [64]. A variety of liposomes of differing compositions were characterized. The best-performing Liposome candidate contained sphingomyelin, 1,2-dimyristoyl-sn-glycero-3-phosphocholine (DMPC), and cholesterol at a 40:40:20% weight to weight ratio. These “SDC” liposomes are well-characterized with a peak mean diameter of 140 nm, a polydispersity index (PDI) of <0.01, and a Zeta potential of +2.32 mV with no significant changes during storage over 3 months at 4° C.

Complexation of SDC Liposomes with a miRNAs (LipomiRs): Addition of a miR-515 agomir to purified SDC liposomes slightly increased their size to −147 nm with PDI of <0.032 and reduced their zeta potential from +2.32 mV to −55.7 mV, indicative of miRNAs surface association. Using high content fluorescence imaging, these SDC liposome miRNA complexes (LipomiRs) showed efficient delivery of fluorescent and functional miRNAs into adipocytes.

Uptake of miRNA was visually confirmed by microscopy along with a dose dependent induction of UCP1 expression seen by qRT-PCR analysis. UCP1 upregulation was analogous to positive control of free miRNA delivered by a DharmaFect transfection reagent.

### VI. REFERENCES

The following references, and those cited elsewhere herein, to the extent that they provide exemplary procedural or other details supplementary to those set forth herein, are specifically incorporated herein by reference.
1 Lheureux, S., M. Braunstein, and A. M. Oza, Epithelial Ovarian Cancer: Evolution of management in the era of precision medicine. CA Cancer J Clin, 2019. 69(4): p. 280-304.
2. Gaona-Luviano, P., L. A. Medina-Gaona, and K. Magana-Perez, Epidemiology of Ovarian Cancer. Chin Clin Oncol, 2020. 9(4): p. 47.
3. Sung, H., et al., Global Cancer Statistics 2020: GLOBOCAN Estimates of Incidence and Mortality Worldwide for 36 Cancers in 185 Countries. CA Cancer J Clin, 2021. 71(3): p. 209-249.
4. Cook, D. P. and B. C. Vanderhyden, Ovarian Cancer and the evolution of subtype classifications using transcriptional profilingdagger. Biol Reprod, 2019. 101(3): p. 645-658.
5. Roett, M. A. and P. Evans, Ovarian Cancer: an overview. Am Fam Physician, 2009. 80(6): p. 609-16.
6. Kossai, M., et al., Ovarian Cancer: A Heterogeneous Disease. Pathobiology, 2018. 85(1-2): p. 41-49.
7 Falzone, L., et al., A multidisciplinary approach remains the best strategy to improve and strengthen the management of Ovarian Cancer (Review). Int J Oncol, 2021. 59(1).
8. Wu, M., et al., Identification of Hub Genes in High-Grade Serous Ovarian Cancer Using Weighted Gene Co-Expression Network Analysis. Med Sci Monit, 2020. 26: p. e922107.
9. Yang, D., et al., Integrated bioinformatics analysis for the screening of hub genes and therapeutic drugs in Ovarian Cancer. J Ovarian Res, 2020. 13(1): p. 10.
10. Yang, Y., et al., Identification of metastasis and prognosis-associated genes for serous Ovarian Cancer. Biosci Rep, 2020. 40(6).
11. Talhouk, A., et al., Development and Validation of the Gene Expression Predictor of High-grade Serous Ovarian Carcinoma Molecular SubTYPE (PrOTYPE). Clin Cancer Res, 2020. 26(20): p. 5411-5423.
12. Pascual-Anton, L., et al., Mesothelial-to-Mesenchymal Transition and Exosomes in Peritoneal Metastasis of Ovarian Cancer. Int J Mol Sci, 2021. 22(21).
13. Yoshida, K., et al., The clinical impact of intra- and extracellular miRNAs in Ovarian Cancer. Cancer Sci, 2020. 111(10): p. 3435-3444.
14. Deb, B., A. Uddin, and S. Chakraborty, miRNAs and Ovarian Cancer: An overview. J Cell Physiol, 2018. 233(5): p. 3846-3854.
15. Mirahmadi, Y., et al., MicroRNAs as Biomarkers for Early Diagnosis, Prognosis, and Therapeutic Targeting of Ovarian Cancer. J Oncol, 2021. 2021: p. 3408937.
16. Strumidlo, A., et al., The potential role of miRNAs in therapy of breast and Ovarian Cancers associated with BRCA1 mutation. Hered Cancer Clin Pract, 2017. 15: p. 15.
17. Croft, P. K., et al., Ovarian-Cancer-Associated Extracellular Vesicles: Microenvironmental Regulation and Potential Clinical Applications. Cells, 2021. 10(9).
18. Ghafouri-Fard, S., H. Shoorei, and M. Taheri, miRNA profile in Ovarian Cancer. Exp Mol Pathol, 2020. 113: p. 104381.
19. Guo, L., et al., Expression profiles analysis reveals an integrated miRNA-lncRNA signature to predict survival in Ovarian Cancer patients with wild-type BRCA1/2. Oncotarget, 2017. 8(40): p. 68483-68492.
20. Hausler, S. F., et al., Whole blood-derived miRNA profiles as potential new tools for Ovarian Cancer screening. Br J Cancer, 2010. 103(5): p. 693-700.
21. Gajek, A., et al., Current Implications of microRNAs in Genome Stability and Stress Responses of Ovarian Cancer. Cancers (Basel), 2021. 13(11).
22. Lopez-Camarillo, C., et al., Deciphering the Long Non-Coding RNAs and MicroRNAs Coregulation Networks in Ovarian Cancer Development: An Overview. Cells, 2021. 10(6).
23. van Rooij, E. and S. Kauppinen, Development of microRNA therapeutics is coming of age. EMBO Mol Med, 2014. 6(7): p. 851-64.
24. Liang, Z., et al., Targeting Membrane Receptors of Ovarian Cancer Cells for Therapy. Curr Cancer Drug Targets, 2019. 19(6): p. 449-467.
25. Della-Longa, S. and A. Arcovito, Structural and functional insights on folate receptor alpha (FRalpha) by homology modeling, ligand docking and molecular dynamics. J Mol Graph Model, 2013. 44: p. 197-207.
26. Scaranti, M., et al., Exploiting the folate receptor alpha in oncology. Nat Rev Clin Oncol, 2020. 17(6): p. 349-359.
27. Wallace-Povirk, A., et al., Folate Transport and One-Carbon Metabolism in Targeted Therapies of Epithelial Ovarian Cancer. Cancers (Basel), 2021. 14(1).
28. Chen, C., et al., Structural basis for molecular recognition of folic acid by folate receptors. Nature, 2013. 500(7463): p. 486-9.
29. Cheung, A., et al., Targeting folate receptor alpha for cancer treatment. Oncotarget, 2016. 7(32): p. 52553-52574.
30. Marchetti, C., et al., Targeted drug delivery via folate receptors in recurrent Ovarian Cancer: a review. Onco Targets Ther, 2014. 7: p. 1223-36.
31. Martin-Sabroso, C., et al., Active Targeted Nanoformulations via Folate Receptors: State of the Art and Future Perspectives. Pharmaceutics, 2021. 14(1).
32. Kalli, K. R., et al., Folate receptor alpha as a tumor target in epithelial Ovarian Cancer. Gynecol Oncol, 2008. 108(3): p. 619-26.
33. Yang, Y., et al., Tumor Microenvironment in Ovarian Cancer: Function and Therapeutic Strategy. Front Cell Dev Biol, 2020. 8: p. 758.
34. Nieman, K. M., et al., Adipocytes promote Ovarian Cancer metastasis and provide energy for rapid tumor growth. Nat Med, 2011. 17(11): p. 1498-503.
35. Motohara, T., et al., An evolving story of the metastatic voyage of Ovarian Cancer cells: cellular and molecular orchestration of the adipose-rich metastatic microenvironment. Oncogene, 2019. 38(16): p. 2885-2898.
36. Ji, Z., et al., Deregulation of Lipid Metabolism: The Critical Factors in Ovarian Cancer. Front Oncol, 2020. 10: p. 593017.
37. Zhao, G., H. Cardenas, and D. Matei, Ovarian Cancer-Why Lipids Matter. Cancers (Basel), 2019. 11(12).
38. Iyoshi, S., et al., Pro-tumoral behavior of omental adipocyte-derived fibroblasts in tumor microenvironment at the metastatic site of Ovarian Cancer. Int J Cancer, 2021. 149(11): p. 1961-1972.
39. Mukherjee, A., et al., Adipocyte-Induced FABP4 Expression in Ovarian Cancer Cells Promotes Metastasis and Mediates Carboplatin Resistance. Cancer Res, 2020. 80(8): p. 1748-1761.
40. Gharpure, K. M., et al., FABP4 as a key determinant of metastatic potential of Ovarian Cancer. Nat Commun, 2018. 9(1): p. 2923.
41. Alexander, R., H. Lodish, and L. Sun, MicroRNAs in adipogenesis and as therapeutic targets for obesity. Expert opinion on therapeutic targets, 2011. 15: p. 623-36.
42. Inui, M., G. Martello, and S. Piccolo, MicroRNA control of signal transduction. Nat Rev Mol Cell Biol, 2010. 11: p. 252-63.
43. Ha, M. and V. N. Kim, Regulation of microRNA biogenesis. Nat Rev Mol Cell Biol, 2014. 15(8): p. 509-24.
44. Li, Z. and T. M. Rana, Therapeutic targeting of microRNAs: current status and future challenges. Nat Rev Drug Discov, 2014. 13(8): p. 622-38.
45. Matsui, M., T. P. Prakash, and D. R. Corey, Argonaute 2-dependent Regulation of Gene Expression by Single-stranded miRNA Mimics. Mol Ther, 2016. 24(5): p. 946-55.
46. Chorn, G., et al., Single-stranded microRNA mimics. RNA, 2012. 18(10): p. 1796-804.
47. Lima, W. F., et al., Single-stranded siRNAs activate RNAi in animals. Cell, 2012. 150(5): p. 883-94.
48. Hu, J., et al., Exploring the effect of sequence length and composition on allele-selective inhibition of human huntingtin expression by single-stranded silencing RNAs. Nucleic Acid Ther, 2014. 24(3): p. 199-209.
49. Kalfert, D., et al., Multifunctional Roles of miR-34a in Cancer: A Review with the Emphasis on Head and Neck Squamous Cell Carcinoma and Thyroid Cancer with Clinical Implications. Diagnostics (Basel), 2020. 10(8).
50. Slabakova, E., et al., Alternative mechanisms of miR-34a regulation in cancer. Cell Death Dis, 2017. 8(10): p. e3100.
51. Glatz, J. F. and J. J. Luiken, From fat to FAT (CD36/SR-B2): Understanding the regulation of cellular fatty acid uptake. Biochimie, 2017. 136: p. 21-26.
52. Zhou, D., et al., CD36 level and trafficking are determinants of lipolysis in adipocytes. FASEB J, 2012. 26(11): p. 4733-42.
53. Luiken, J. J., et al., Post-translational modifications of CD36 (SR-B2): Implications for regulation of myocellular fatty acid uptake. Biochim Biophys Acta, 2016. 1862(12): p. 2253-2258.
54. Zatsepin, T. S., Y. V. Kotelevtsev, and V. Koteliansky, Lipid nanoparticles for targeted siRNA delivery-going from bench to bedside. Int J Nanomedicine, 2016. 11: p. 3077-86.
55. Hardee, G. E., Oral delivery of nucleic acid-based therapeutics. Ther Deliv, 2012. 3(2): p. 143-5.
56. Maher, S., et al., Safety and efficacy of sodium caprate in promoting oral drug absorption: from in vitro to the clinic. Adv Drug Deliv Rev, 2009. 61(15): p. 1427-49.
57. Mosa, R., et al., Hexarelin, a Growth Hormone Secretagogue, Improves Lipid Metabolic Aberrations in Nonobese Insulin-Resistant Male MKR Mice. Endocrinology, 2017. 158(10): p. 3174-3187.
58. Rodrigue-Way, A., et al., A growth hormone-releasing peptide promotes mitochondrial biogenesis and a fat burning-like phenotype through scavenger receptor CD36 in white adipocytes. Endocrinology, 2007. 148(3): p. 1009-18.
59. Yu, B., et al., Insight into Mechanisms of Cellular Uptake of Lipid Nanoparticles and Intracellular Release of Small RNAs. Pharm Res, 2014.
60. Dong, Y., et al., Lipopeptide nanoparticles for potent and selective siRNA delivery in rodents and nonhuman primates. Proc Natl Acad Sci USA, 2014. 111(11): p. 3955-60.
61. Dahlman, J. E., et al., In vivo endothelial siRNA delivery using polymeric nanoparticles with low molecular weight. Nat Nanotechnol, 2014.
62. Milhas, D., C. J. Clarke, and Y. A. Hannun, Sphingomyelin metabolism at the plasma membrane: implications for bioactive sphingolipids. FEBS Lett, 2010. 584(9): p. 1887-94.
63. Lajoie, P., et al., Lattices, rafts, and scaffolds: domain regulation of receptor signaling at the plasma membrane. J Cell Biol, 2009. 185(3): p. 381-5.
64. Webb, M. S., et al., Sphingomyelin-cholesterol liposomes significantly enhance the pharmacokinetic and therapeutic properties of vincristine in murine and human tumour models. Br J Cancer, 1995. 72(4): p. 896-904.

1. A therapeutic agent comprising:
(a) a miRNA oligonucleotide therapeutic; and
(b) a targeting element that binds to an ovarian cancer cell or a cell of an ovarian cancer tumor microenvironment.

2. The therapeutic agent of claim 1, wherein the targeting element binds to an ovarian cancer cell.

3. The therapeutic agent of claim 1, wherein the targeting element binds to a cell of an ovarian cancer tumor microenvironment.

4. The therapeutic agent of claim 1, wherein the cell of the ovarian cancer tumor microenvironment is an adipocyte.

5. The therapeutic agent of claim 1, wherein the miRNA oligonucleotide therapeutic and the targeting element are connected by a linker.

6. The therapeutic agent of claim 1, wherein the miRNA oligonucleotide therapeutic is from 7 to 23 nucleotides in length.

7. The therapeutic agent of claim 1, wherein the miRNA oligonucleotide therapeutic is a miRNA antagomir or agomir.

8. The therapeutic agent of claim 1, wherein the targeting element is folic acid.

9. The therapeutic agent of claim 8, wherein the targeting element binds to folic receptor alpha (FOLR1).

10. The therapeutic agent of claim 1, wherein the targeting element is a peptide.

11. The therapeutic agent of claim 10, wherein the peptide binds to the folic receptor alpha (FOLR1).

12. The therapeutic agent of claim 1, wherein the targeting element is a fatty acid.

13. The therapeutic agent of claim 12, wherein the fatty acid binds to the Fatty Acid Translocase (FAT/CD36/SCARB3) and/or the fatty Acid Binding Protein 4 (FABP4) transporters.

14. The therapeutic agent of claim 1, wherein the targeting element is a peptide.

15. The therapeutic agent of claim 14, wherein the peptide binds to the Fatty Acid Translocase (FAT/CD36/SCARB3) and/or the fatty Acid Binding Protein 4 (FABP4) transporters.

16. The therapeutic agent of claim 5, wherein the linker is a covalent bond, a disulfide bond, a diester bond, a peptide bond, an ionic bond, or a biotin-streptavidin linker.

17. The therapeutic agent of claim 5, wherein the linker is a cleavable linker.

18. The therapeutic agent of claim 5, wherein the linker is a non-cleavable linker.

19. The therapeutic agent of claim 5, wherein the linker is a peptide linker.

20. The therapeutic agent of claim 1, wherein the therapeutic agent is encapsulated within the interior of a lipid nanoparticle.

21. The therapeutic agent of claim 1, wherein the therapeutic agent is associated with the surface of a liposome.

22. A method for treating cancer comprising providing to the subject a therapeutically effective amount of the therapeutic agent of claim 1.

23. The method of claim 22, wherein providing the therapeutic agents is provided subcutaneously, transcutaneously, intraperitoneally, or intravenously.

24. The method of claim 22 or 23, wherein the subject has Ovarian Cancer.


SEQUENCE LISTING (7 sequences)

SEQ ID NO: 1 - protein, 8 aa, Artificial Sequence
  Synthetic Polypeptide
LSCQLYQR

SEQ ID NO: 2 - protein, 9 aa, Artificial Sequence
  Synthetic Polypeptide
CTVRTSADC

SEQ ID NO: 3 - protein, 12 aa, Artificial Sequence
  Synthetic Polypeptide
DWSSWVYRDPQT

SEQ ID NO: 4 - protein, 12 aa, Artificial Sequence
  Synthetic Polypeptide
SGVYKVAYDWQH

SEQ ID NO: 5 - protein, 9 aa, Artificial Sequence
  Synthetic Polypeptide
CIGNSNTLC

SEQ ID NO: 6 - protein, 9 aa, Artificial Sequence
  Synthetic Polypeptide
CTVRTSAEC

SEQ ID NO: 7 - protein, 12 aa, Artificial Sequence
  Synthetic Polypeptide
MHTAPGWGYRLS